(19) United States
(12) Patent Application Publication
(10) Pub. No.: US 2012/0190613 A1
(43) Pub. Date: Jul. 26, 2012

(54) ECHINOCANDIN DERIVATIVES

(76) Inventors: Kenneth Duke James, JR., Mebane, NC (US); Christopher Patrick Laudeman, Durham, NC (US)

(21) Appl. No.: 13/392,152

(22) PCT Filed: Aug. 26, 2010
(86) PCT No.: PCT/US10/46800

§ 371 Date: Mar. 21, 2012

Related U.S. Application Data
(60) Provisional application No. 61/237,427, filed on Aug. 27, 2009.

(51) Int. Cl. A61K 38/12; A01P 3/00; A01N 37/46; C07K 7/56; A61P 31/10

(52) U.S. Cl. 514/3.4; 530/317; 514/3.6; 514/21.1

(57) ABSTRACT

The invention features echinocandin class compounds that have been modified to (i) have activity against one or more fungal species or genera; (ii) have increased aqueous solubility; (iii) have an increased therapeutic index; (iv) be suitable for topical administration; and/or (v) be suitable for oral administration. The echinocandin class compounds of the invention include, for example, a PEG, alkyl-PEG, aryl-PEG, alkaryl-PEG, PEG-alkyl, PEG-aryl, or PEG-alkaryl substituent.

### CROSS-REFERENCE TO RELATED APPLICATIONS

This application claims benefit of U.S. Provisional Application No. 61/237,427, filed Aug. 27, 2009, which is incorporated herein by reference.

### BACKGROUND OF THE INVENTION

This invention relates to the field of treatment of fungal infections.

The need for novel antifungal treatments is significant, and is especially critical in the medical field. Immunocompromised patients provide perhaps the greatest challenge to modern health care delivery. During the last three decades there has been a dramatic increase in the frequency of fungal infections in these patients (Herbrecht, Eur. J. Haematol., 56:12, 1996; Cox et al., Curr. Opin. Infect. Dis., 6:422, 1993; Fox, ASM News, 59:515, 1993). Deep-seated mycoses are increasingly observed in patients undergoing organ transplants and in patients receiving aggressive cancer chemotherapy (Alexander et al., Drugs, 54:657, 1997). The most common pathogens associated with invasive fungal infections are the opportunistic yeast, Candida albicans, and the filamentous fungus, Aspergillus fumigatus (Bow, Br. J. Haematol., 101:1, 1998; Wamock, J. Antimicrob. Chemother., 41:95, 1998). There are an estimated 200,000 patients per year who acquire nosocomial fungal infections (Beck-Sague et al., J. Infect. Dis., 167:1247, 1993). Also adding to the increase in the numbers of fungal infections is the emergence of Acquired Immunodeficiency Syndrome (AIDS) where virtually all patients become affected with some form of mycoses during the course of the disease (Alexander et al., Drugs, 54:657, 1997; Hood et al., J. Antimicrob. Chemother., 37:71, 1996). The most common organisms encountered in these patients are Cryptococcus neoformans, Pneumocystis carinii, and C. albicans (HIV/AIDS Surveillance Report, 1996, 7(2), Year-End Edition; Polis, M. A. et al., AIDS: Biology, Diagnosis, Treatment and Prevention, fourth edition, 1997). New opportunistic fungal pathogens such as Penicillium marneffei, C. krusei, C. glabrata, Histoplasma capsulatum, and Coccidioides immitis are being reported with regularity in immunocompromised patients throughout the world.

The development of antifungal treatment regimens has been a continuing challenge. Currently available drugs for the treatment of fungal infections include amphotericin B, a macrolide polyene that interacts with fungal membrane sterols, flucytosine, a fluoropyrimidine that interferes with fungal protein and DNA biosynthesis, and a variety of azoles (e.g., ketoconazole, itraconazole, and fluconazole) that inhibit fungal membrane-sterol biosynthesis (Alexander et al., Drugs, 54:657, 1997). Even though amphotericin B has a broad range of activity and is viewed as the “gold standard” of antifungal therapy, its use is limited due to infusion-related reactions and nephrotoxicity (Wamock, J. Antimicrob. Chemother., 41:95, 1998). Flucytosine usage is also limited due to the development of resistant microbes and its narrow spectrum of activity. The widespread use of azoles is causing the emergence of clinically-resistant strains of Candida spp. Due to the problems associated with the current treatments, there is an ongoing search for new treatments.

When the echinocandin caspofungin was approved for sale in 2002, it represented the first new class of antifungal agents to be approved in over a decade. Since that time, two other echinocandin antifungals, anidulafungin and micafungin, have been approved in various markets. Each agent in this class of compound acts by inhibition of β-1,3-glucan synthase, which is a key enzyme in the synthesis of glucan in the cell wall of many fungi. All three of these drugs are made semisynthetically starting with a natural product obtained through fermentation.

The echinocandins are a broad group of antifungal agents that typically are comprised of a cyclic hexapeptide and lipophilic tail, the latter of which is attached to the hexapeptide core through an amide linkage. Although many echinocandins are natural products, the clinically relevant members of this class have all been semisynthetic derivatives. Although the naturally occurring echinocandins possess anti-fungal activity, they have not been suitable as therapeutics, primarily because of poor aqueous solubility and/or hemolytic action. The approved echinocandins are the products of intense efforts to generate derivatives that maintain the glucan synthase inhibition, but do not cause the hemolytic effects. As therapeutic agents, they are attractive compounds in terms of their systemic half-lives, large therapeutic windows, safety profiles, and relative lack of interactions with other drugs. Unfortunately, the poor aqueous solubility and poor intestinal absorption of these compounds have relegated them to delivery by intravenous infusion. Although patients receiving these drugs are often hospitalized with serious infections, the ability to transition patients from intravenous delivery in a hospital setting to oral delivery in a home setting would be very desirable, especially considering the course of the regimen commonly exceeds 14 days. In addition, an oral echinocandin may expand the use of this drug class to include patients that present with mild fungal infections.

The present invention features derivatives of echinocandin antifungals that can have increased aqueous solubility and/or are suitable for oral delivery.

### SUMMARY OF THE INVENTION

The invention features echinocandin class compounds that have been modified to (i) have activity against one or more fungal species or genera; (ii) have increased aqueous solubility; (iii) have an increased therapeutic index; (iv) be suitable for topical administration; and/or (v) be suitable for oral administration. The echinocandin class compounds of the invention include, for example, a PEG, alkyl-PEG, aryl-PEG, alkaryl-PEG, PEG-alkyl, PEG-aryl, or PEG-alkaryl substituent.

In a first aspect the invention features an echinocandin class compound including a PEG, alkyl-PEG, aryl-PEG, alkaryl-PEG, PEG-alkyl, PEG-aryl, or PEG-alkaryl group.

The echinocandin class compound can be described by formula (I):

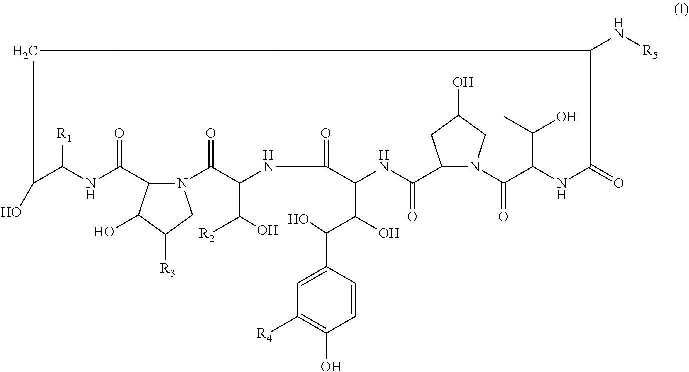

In formula (I), R¹ is NHCH₂CH₂NHR^{A1}, NHCH₂CH₂NR^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1}; R² is H, CH₃, CH₂CH₂NHR^{B1}, CH₂CH₂NR^{B1}R^{B2}, CH₂CH₂NHC(O)R^{B1}, CH₂C(O)NHR^{B1}, CH₂CH₂CH(OR^{B1})NHR^{B2}, CH₂CH₂CH(OR^{B1})NR^{B2}R^{B3}, or CH₂CH₂CH(OR^{B1})NHC(O)R^{B2}; R³ is H or CH₃; R⁴ is H, OSO₃H, CH₂NHR^{C1}, CH₂NR^{C1}R^{C2}, CH₂NHC(O)R^{C1}; R⁵ is a lipophilic group selected from: PEG; C(O)-PEG; PEG-alkyl; C(O)-PEG-alkyl; PEG-aryl; C(O)-PEG-aryl; PEG-alkaryl; C(O)-PEG-alkaryl; alkyl-PEG; C(O)-alkyl-PEG; aryl-PEG; C(O)-aryl-PEG; alkaryl-PEG; C(O)-alkaryl-PEG;

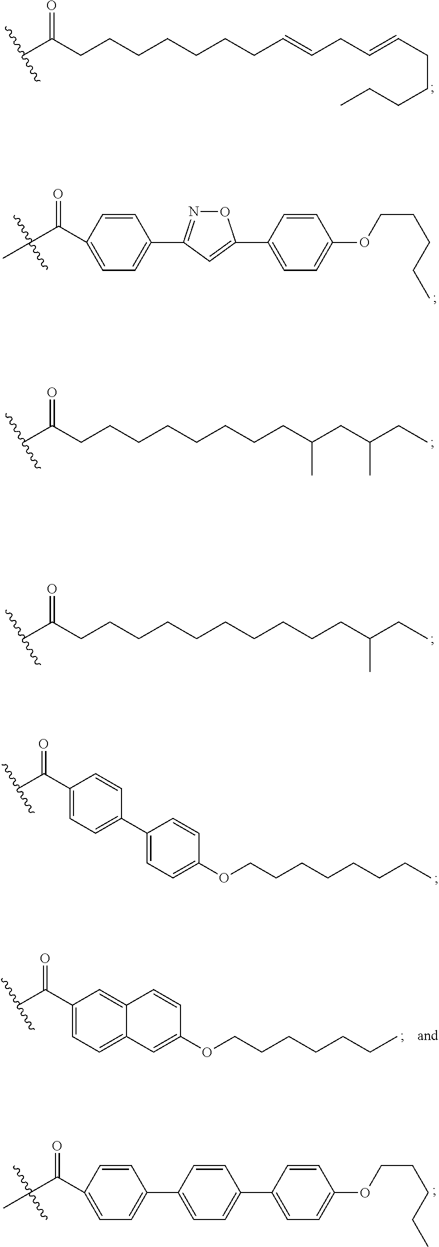

and each of R^{A1}, R^{A2}, R^{B1}, R^{B2}, R^{B3}, R^{C1}, and R^{C2} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl, and pharmaceutically acceptable salts thereof, provided that the echinocandin class compound includes at least one PEG, alkyl-PEG, aryl-PEG, alkaryl-PEG, PEG-alkyl, PEG-aryl, or PEG-alkaryl group.

The echinocandin class compound of formula (I) can further be described by formula (II):

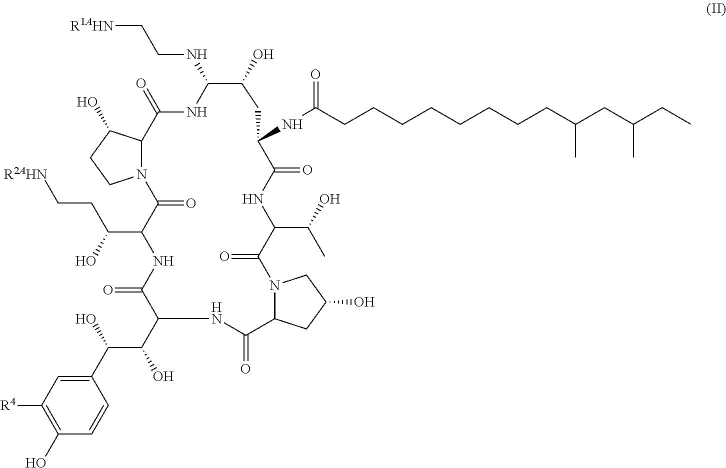

In formula (II), R^{1A} is H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ hctcroalkyl, PEG, alkyl-PEG, aryl-PEG, alkaryl-PEG, PEG-alkyl, PEG-aryl, or PEG-alkaryl; R^{2A} is H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, or PEG-alkaryl; R⁴ is H, OSO₃H, CH₂NHR^{C1}, CH₂NR^{C1}R^{C2}, CH₂NHC(O)R^{C1}; and each of R^{C1} and R^{C2} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl, and pharmaceutically acceptable salts thereof, provided that the echinocandin class compound includes at least one PEG, alkyl-PEG, aryl-PEG, alkaryl-PEG, PEG-alkyl, PEG-aryl, or PEG-alkaryl.

In certain embodiments of the echinocandin class compound of formula (II), one of R^{1A}, R^{2A}, R^{C1} and R^{C2} is selected from: (i) —(CH₂)ₚ—O—(CH₂CH₂O)ₘ—Me, and (ii) —(CH₂CH₂O)ₘ—Me, and (iii) —C(O)(CH₂)ₙ—(OCH₂CH₂)ₘ—OMe, wherein n is an integer from 0 to 11 (e.g., 0 to 7, 1 to 7, 2 to 7, 3 to 9, or 4 to 11), p is an integer from 3 to 12 (e.g., 3 to 8, 4 to 10, or 6 to 12), and m is an integer from 1 to 10 (e.g., 1 to 7, 1 to 5, 2 to 7, 2 to 5, or 3 to 7). In particular embodiments of the echinocandin class compound of formula (II), R^{1A} is H and R^{2A} is PEG, alkyl-PEG, aryl-PEG, alkaryl-PEG, PEG-alkyl, PEG-aryl, or PEG-alkaryl; R^{1A} is PEG, alkyl-PEG, aryl-PEG, alkaryl-PEG, PEG-alkyl, PEG-aryl, or PEG-alkaryl and R^{2A} is H; or each of R^{1A} and R^{2A} is, independently, selected from PEG, alkyl-PEG, aryl-PEG, alkaryl-PEG, PEG-alkyl, PEG-aryl, and PEG-alkaryl.

The echinocandin class compound of formula (I) can further be described by formula (III):

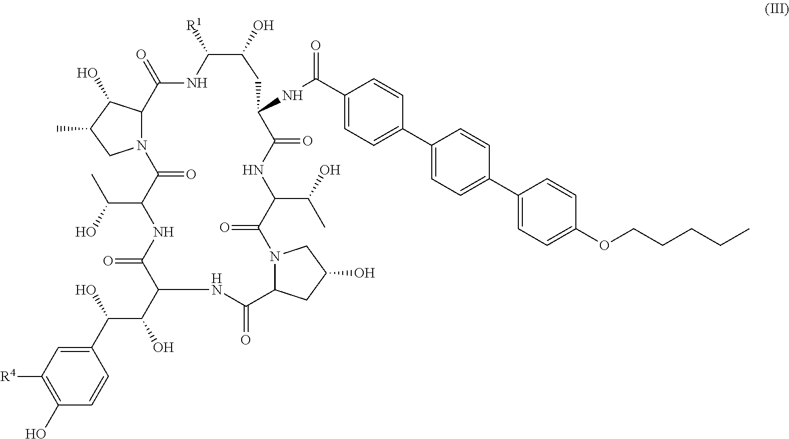

In formula (III), R′ is NHCH₂CH₂NHR^{A1}, NHCH₂CH₂^{NR}^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1}; R⁴ is H, OSO₃H, CH₂NHR^{C1}, CH₂CH₂NHC(O)R^{C1}; and each of R^{A1}, R^{A2}, R^{C1}, and R^{C2} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl, and pharmaceutically acceptable salts thereof, provided that the echinocandin class compound includes at least one PEG, alkyl-PEG, aryl-PEG, alkaryl-PEG, PEG-alkyl, PEG-aryl, or PEG-alkaryl group.

In certain embodiments of the echinocandin class compound of formula (III), R¹ is selected from: (i) —O—(CH₂CH₂O)ₘ—(CH₂)—Me, (ii) —NH—(CH₂CH₂O)ₘ—(CH₂)ₙ—Me, (iii) —O—(CH₂)_{q}—O—(CH₂CH₂O)ₘ—Me, (iv) —NH—(CH₂)_{q}—O—(CH₂CH₂O)ₘ—Me, (v) —O—(CH₂)ₚ—NH—(CO)—(CH₂)ₙ—O—(CH₂CH₂O)ₘ—Me, (vi) —NH—(CH₂)ₚ—NH—(CO)—(CH₂)ₙ—O—(CH₂CH₂O)ₘ—Me, (vii) —NHCH[(CH₂O(CH₂CH₂O)ₛ—Me)(CH₂O(CH₂CH₂O)ₜ—Me)], and (viii) —O—CH[(CH₂O(CH₂CH₂O)ₛ—Me)(CH₂O(CH₂CH₂O)ₜ—Me)], wherein n is an integer from 0 to 11 (e.g., 0 to 7, 1 to 7, 2 to 7, 3 to 9, or 4 to 11), q is an integer from 3 to 12 (e.g., 3 to 7, 5 to 9, or 7 to 12), p is an integer from 2 to 8 (e.g., 2 to 4, 3 to 6, or 4 to 8), s is an integer from 0 to 5 (e.g., 0, 1, 2, 3, 4, or 5), t is an integer from 0 to 5 (e.g., 0, 1, 2, 3, 4, or 5), and m is an integer from 1 to 10 (e.g., 1 to 7, 1 to 5, 2 to 7, 2 to 5, or 3 to 7).

The echinocandin class compound of formula (I) can further be described by formula (IV):

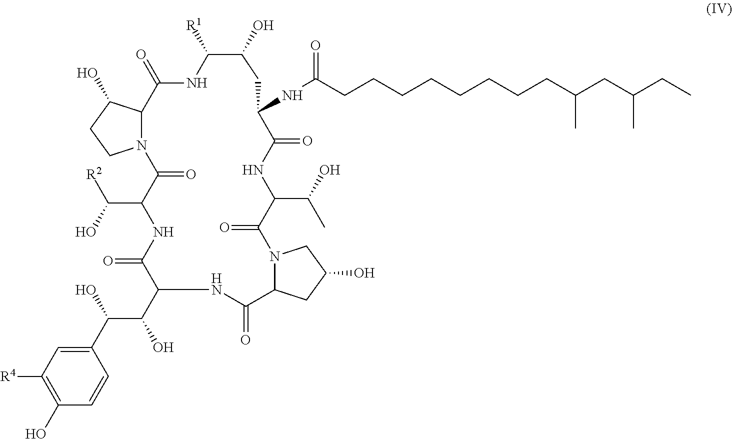

In formula (IV), R¹ is NHCH₂CH₂NHR^{A1}, NHCH₂CH₂NR^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1}; R² is H, CH₃, CH₂CH₂NHR^{B1}, CH₂CH₂NR^{B1}R^{B2}, CH₂CH₂NHC(O)R^{B1}, CH₂C(O)NHR^{B1}, CH₂CH₂CH(OR^{B1})NHR^{B2}, CH₂CH₂CH(OR^{B1})NR^{B2}R^{B3}, or CH₂CH₂CH(OR^{B1})NHC(O)R^{B2}; R⁴ is H, OSO₃H, CH₂NHR^{C1}, CH₂NR^{C1}R^{C2}, CH₂NHC(O)R^{C1}; and each of R^{A1}, R^{A2}, R^{B1}, R^{B2}, R^{B3}, R^{C1}, and R^{C2} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl, and pharmaceutically acceptable salts thereof, provided that the echinocandin class compound includes at least one PEG, alkyl-PEG, aryl-PEG, alkaryl-PEG, PEG-alkyl, PEG-aryl, or PEG-alkaryl group.

In certain embodiments of the echinocandin class compound of formula (IV), R¹ is selected from: (i) —O—(CH₂CH₂O)ₘ—(CH₂)ₙ—Me, (ii) —NH—(CH₂CH₂O)ₘ—(CH₂)ₙ—Me, (iii) —O—(CH₂)_{q}—O—(CH₂CH₂O)ₘ—Me, (iv) —NH—(CH₂)_{q}—O—(CH₂CH₂O)ₘ—Me, (v) —O—(CH₂)ₚ—NH—(CO)—(CH₂)ₙ—O—(CH₂CH₂O)ₘ—Me, (vi) —NH—(CH₂)ₚ—NH—(CO)—(CH₂)ₙ—O—(CH₂CH₂O)ₘ—Me, (vii) —NHCH[(CH₂O(CH₂CH₂O)ₛ—Me)(CH₂O(CH₂CH₂O)ₜ—Me)], and (viii) —O—CH[(CH₂O(CH₂CH₂O)ₛ—Me)(CH₂O(CH₂CH₂O)ₜ—Me)], wherein n is an integer from 0 to 11 (e.g., 0 to 7, 1 to 7, 2 to 7, 3 to 9, or 4 to 11), q is an integer from 3 to 12 (e.g., 3 to 7, 5 to 9, or 7 to 12), p is an integer from 2 to 8 (e.g., 2 to 4, 3 to 6, or 4 to 8), s is an integer from 0 to 5 (e.g., 0, 1, 2, 3, 4, or 5), t is an integer from 0 to 5 (e.g., 0, 1, 2, 3, 4, or 5), and m is an integer from 1 to 10 (e.g., 1 to 7, 1 to 5, 2 to 7, 2 to 5, or 3 to 7 ).

The echinocandin class compound of formula (I) can further be described by formula (V):

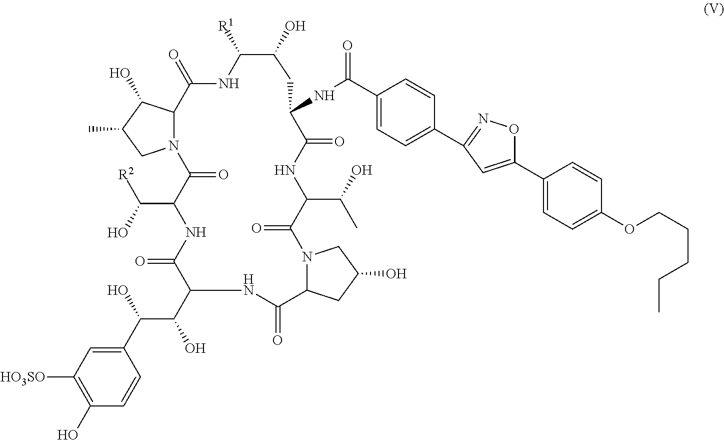

In formula (V), R¹ is NHCH₂CH₂NHR^{A1}, NHCH₂CH₂NR^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1}; R² is H, CH₃, CH₂CH₂NHR^{B1}, CH₂CH₂NR^{B1}R^{B2}, CH₂CH₂NHC(O)R^{B1}, CH₂C(O)NHR^{B1}, CH₂CH₂CH(OR^{B1})NHR^{B2}, CH₂CH₂CH(OR^{B1})NR^{B2}R^{B3}, or CH₂CH₂CH(OR^{B1})NHC(O)R^{B2}; and each of R^{A1}, R^{A2}, R^{B1}, R^{B2}, and R^{B3} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl, and pharmaceutically acceptable salts thereof, provided that the echinocandin class compound includes at least one PEG, alkyl-PEG, aryl-PEG, alkaryl-PEG, PEG-alkyl, PEG-aryl, or PEG-alkaryl group.

In certain embodiments of the echinocandin class compound of formula (V), R¹ is selected from: (i) —O—(CH₂CH₂O)ₘ—(CH₂)ₙ—Me, (ii) —NH—(CH₂CH₂O)ₘ—(CH₂)ₙ—Me, (iii) —O—(CH₂)_{q}—O—(CH₂CH₂O)ₘ—Me, (iv) —NH—(CH₂)_{q}—O—(CH₂CH₂O)ₘ—Me, (v) —O—(CH₂)ₚ—NH—(CO)—(CH₂)ₙ—O—(CH₂CH₂O)ₘ—Me, (vi) —NH—(CH₂)ₚ—NH—(CO)—(CH₂)—O—(CH₂CH₂O)ₘ—Me, (vii) —NHCH[(CH₂O(CH₂CH₂O)ₛ—Me)(CH₂O(CH₂CH₂O)ₜ—Me)], and (viii) —O—CH[(CH₂O(CH₂CH₂O)ₛ—Me)(CH₂O(CH₂CH₂O)ₜ—Me)], wherein n is an integer from 0 to 11 (e.g., 0 to 7, 1 to 7, 2 to 7, 3 to 9, or 4 to 11), q is an integer from 3 to 12 (e.g., 3 to 7, 5 to 9, or 7 to 12), p is an integer from 2 to 8 (e.g., 2 to 4, 3 to 6, or 4 to 8), s is an integer from 0 to 5 (e.g., 0, 1, 2, 3, 4, or 5), t is an integer from 0 to 5 (e.g., 0, 1, 2, 3, 4, or 5), and m is an integer from 1 to 10 (e.g., 1 to 7, 1 to 5, 2 to 7, 2 to 5, or 3 to 7).

In particular embodiments of the echinocandin class compound of formula (I), R⁴ is selected from: (i) —CH₂NH—(CH₂CH₂O)ₘ—(CH₂)ₙ—Me, (ii) —CH₂NH—(CH₂)_{q}—O—(CH₂CH₂O)ₘ—Me, (iii) —CH₂NH—(CH₂)ₚ—NH—(CO)—(CH₂)ₙ—O—(CH₂CH₂O)ₘ—Me, and (iv) —CH₂NHCH[CH₂O(CH₂CH₂O)ₛ—Me)(CH₂O(CH₂CH₂O)ₜ—Me)], wherein n is an integer from 0 to 11 (e.g., 0 to 7, 1 to 7, 2 to 7, 3 to 9, or 4 to 11), q is an integer from 3 to 12 (e.g., 3 to 7, 5 to 9, or 7 to 12), p is an integer from 2 to 8 (e.g., 2 to 4, 3 to 6, or 4 to 8), s is an integer from 0 to 5 (e.g., 0, 1, 2, 3, 4, or 5), t is an integer from 0 to 5 (e.g., 0, 1, 2, 3, 4, or 5), and m is an integer from 1 to 10 (e.g., 1 to 7, 1 to 5, 2 to 7, 2 to 5, or 3 to 7).

In still other embodiments of the echinocandin class compound of formula (I), R⁵ is selected from: (i) —(CH₂CH₂O)ₘ—(CH₂)ₙ—Me, (ii) —C(O)—(CH₂CH₂O)ₘ—(CH₂)ₙ—Me, (iii) —C(O)CH₂—O—(CH₂CH₂O)ₘ—(CH₂)ₙ—Me, and (iv) —C(O)—O—(CH₂CH₂O)ₘ—(CH₂)ₙ—Me, wherein n is an integer from 0 to 11 (e.g., 0 to 7, 1 to 7, 2 to 7, 3 to 9, or 4 to 11), and m is an integer from 1 to 10 (c.g., 1 to 7, 1 to 5, 2 to 7, 2 to 5, or 3 to 7).

In certain embodiments, the echinocandin class compound of the invention (i) has increased oral bioavailability; (ii) has increased transdermal bioavailability; and/or (iii) has an increased therapeutic index.

In a related aspect, the invention features a pharmaceutical composition including an echinocandin class compound of the invention, or a salt thereof, and a pharmaceutically acceptable excipient. In certain embodiments, the pharmaceutical composition includes a mixture of echinocandin class compounds which are substantially monodisperse.

The echinocandin class compound of the invention can be formulated for oral administration in unit dosage form, or formulated and administered in any manner described herein.

The invention also features a method of treating a fungal infection in a subject by administering to the subject an echinocandin class compound of the invention, or a salt thereof, in an amount sufficient to treat the infection.

Fungal infections which can be treated using echinocandin class compounds of the invention include, without limitation, tinea capitis, tinea corporis, tinea pedis, onychomycosis, perionychomycosis, pityriasis versicolor, oral thrush, vaginal candidosis, respiratory tract candidosis, biliary candidosis, eosophageal candidosis, urinary tract candidosis, systemic candidosis, mucocutaneous candidosis, aspergillosis, mucormycosis, paracoccidioidomycosis, North American blastomycosis, histoplasmosis, coccidioidomycosis, and sporotrichosis. For example, the echinocandin class compounds of the invention can be used to treat a fungal infection of Candida albicans, C. parapsilosis, C. glabrata, C. guilliermondii, C. krusei, C. tropicalis, Aspergillus fumigatus, A. flavus, or A. terreus.

The echinocandin class compound of the invention can be administered orally, or in any manner of administration described herein.

The invention features a method of preventing, stabilizing, or inhibiting the growth of fungi, or killing fungi, by contacting the fungi or a site susceptible to fungal growth (e.g., shoes, bathroom tiles, shower curtains, etc.) with an echinocandin class compound of the invention, or a salt thereof.

The echinocandin class compounds and pharmaceutical compositions of the invention are, optionally, deuterated compounds in which one or more hydrogen atoms of the echinocandin class compound is isotopically enriched (e.g., 85%, 90%, 95%, or 98%) with deuterium.

As used herein, the term “echinocandin class compound” refers to an antibiotic cyclic lipohexapeptide which is an inhibitor of the synthesis of 1,3-β-D-glucan. Echinocandin class compounds including a backbone shown below.

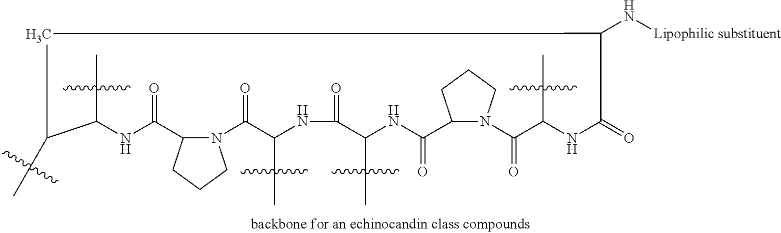

Echinocandin class compounds include, without limitation, caspofungin, echinocandin B, anidulafungin, pneumocandin B₀, aculeacin A_{γ}, and micafungin.

In the generic descriptions of compounds of this invention, the number of atoms of a particular type in a substituent group is generally given as a range. Reference to such a range is intended to include specific references to groups having each of the integer number of atoms within the specified range.

For example, an alkyl group from 1 to 10 carbon atoms includes each of C₁, C₂, C₃, C₄, C₅, C₆, C₇, C₈, C₉, and C₁₀. Other numbers of atoms and other types of atoms are indicated in a similar manner.

By “PEG” is meant a group having the formula A-B-(C)_{q}, wherein A is absent, NH, or O; B is (CH₂CH₂O)ₘ or CH[(CH₂O(CH₂CH₂O)ₚ)(CH₂O(CH₂CH₂O)ᵣ)] wherein in is an integer from 1 to 10, p is an integer from 0 to 5, and r is an integer from 0 to 5; C is a terminal group that is H, CH₃, or CH₂CH₃; and q is 1 or 2.

By “alkyl-PEG” is meant a group having the formula A-B-(C)_{q}, wherein A is C₃₋₁₅ alkyl, C₃₋₁₅ alkenyl, C₃₋₁₅ alkynyl, or C₃₋₁₅ heteroalkyl; B is X(CH₂CH₂O)ₘ or XCH[(CH₂O(CH₂CH₂O)ₚ)(CH₂O(CH₂CH₂O)ᵣ)] wherein m is an integer from 1 to 10, p is an integer from 0 to 5, r is an integer from 0 to 5, and X is O or NH; C is a terminal group that is H or an organic radical of less than 100 Daltons, such as CH₃, or CH₂CH₃; and q is 1 or 2.

By “aryl-PEG” is meant a group having the formula A-B-(C)_{q}, wherein A is C₆₋₁₈ aryl; B is X(CH₂CH₂O)ₘ or XCH[(CH₂O(CH₂CH₂O)ₚ)(CH₂O(CH₂CH₂O)ᵣ)] wherein m is an integer from 1 to 10, p is an integer from 0 to 5, r is an integer from 0 to 5, and X is O or NH; C is a terminal group that is H or an organic radical of less than 100 Daltons, such as CH₃, or CH₂CH₃; and q is 1 or 2.

By “alkaryl-PEG” a group having the formula A-B-(C)_{q}, wherein A is a C₁₋₁₅ alkyl, C₂₋₁₅ alkenyl, C₂₋₁₅ alkynyl, or C₁₋₁₅ heteroalkyl group substituted by a C₆₋₁₈ aryl (e.g., phenyl, biphenyl, or triphenyl); B is X(CH₂CH₂O)ₙ, or XCH[(CH₂O(CH₂CH₂O)ₚ)(CH₂O(CH₂CH₂O)ᵣ)] wherein m is an integer from 1 to 10, p is an integer from 0 to 5, r is an integer from 0 to 5, and X is O or NH; C is a terminal group that is H or an organic radical of less than 100 Daltons, such as CH₃, or CH₂CH₃; and q is 1 or 2.

By “PEG-alkyl” is meant a group having the formula A-B-(C)₄, wherein A is absent, O, or NH; B is (CH₂CH₂O)ₘ or CH[(CH₂O(CH₂CH₂O)ₚ)(CH₂O(CH₂CH₂O)ᵣ)] wherein m is an integer from 1 to 10, p is an integer from 0 to 5, and r is an integer from 0 to 5; C is C₁₋₁₅ alkyl, C₂₋₄₅ alkenyl, C₂₋₁₅ alkynyl, or C₁₋₁₅ heteroalkyl; and q is 1 or 2.

By “PEG-aryl” is meant a group having the formula A-B-(C)_{q}, wherein A is absent, O, or NNH; B is (CH₂CH₂O)ₘ or CH[(CH₂O(CH₂CH₂O)ₚ)(CH₂O(CH₂CH₂O)ᵣ)] wherein in is an integer from 1 to 10, p is an integer from 0 to 5, and r is an integer from 0 to 5; C is C₆₋₁₈ aryl;

and q is 1 or 2.

By “PEG-alkaryl” a group having the formula A-B-(C)_{q}, wherein A is absent, O, or NH; B is (CH₂CH₂O)ₘ or CH[(CH₂O(CH₂CH₂O)ₚ)(CH₂O(CH₂CH₂O)ᵣ)] wherein m is an integer from 1 to 10, p is an integer from 0 to 5, and r is an integer from 0 to 5; C is C_{1-15 alkyl, C}₂₋₁₅ alkenyl, C₂₋₁₅ alkynyl, or C₁₋₁₅ heteroalkyl group substituted by a C₆₋₁₈ aryl (e.g., phenyl, biphenyl, or triphenyl); and q is 1 or 2.

By “substantially monodisperse” is meant a mixture of echinocandin class compounds wherein at least about 90%, 95%, or 98% of the echinocandin class compounds in the mixture have the same molecular weight.

By “C₁₋₁₀ alkyl” is meant a branched or unbranched hydrocarbon group having from 1 to 10 carbon atoms. A C₁₋₁₀ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, carboxyl, PEG, alkyl-PEG, aryl-PEG, alkaryl-PEG, PEG-alkyl, PEG-aryl, and PEG-alkaryl groups. C₁₋₁₀ alkyls include, without limitation, methyl, ethyl, n-propyl, isopropyl, cyclopropyl, cyclopropylmethyl, n-butyl, iso-butyl, sec-butyl, tert-butyl, cyclobutyl, n-pentyl, cyclopentyl, n-hexyl, cyclohexyl, heptyl, and octyl, among others. Alkyl groups of other lengths are similarly branched or unbranched and substituted or unsubstituted.

By “C₂₋₁₀ alkenyl” is meant a branched or unbranched hydrocarbon group containing one or more double bonds and having from 2 to 10 carbon atoms. A C₂₋₁₀ alkenyl may optionally include monocyclic or polycyclic rings, in which each ring desirably has from three to six members. The C₂₋₁₀ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, carboxyl, PEG, alkyl-PEG, aryl-PEG, alkaryl-PEG, PEG-alkyl, PEG-aryl, and PEG-alkaryl groups. C₂₋₁₀ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl. Alkenyl groups of other lengths are similarly branched or unbranched and substituted or unsubstituted.

By “C₂₋₁₀ alkynyl” is meant a branched or unbranched hydrocarbon group containing one or more triple bonds and having from 2 to 10 carbon atoms. A C₂₋₁₀ alkynyl may optionally include monocyclic, bicyclic, or tricyclic rings, in which each ring desirably has five or six members. The C₂₋₁₀ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, carboxyl, PEG, alkyl-PEG, aryl-PEG, alkaryl-PEG, PEG-alkyl, PEG-aryl, and PEG-alkaryl groups. C₂₋₁₀ alkynyls include, without limitation, ethynyl, 1-propynyl, 2-propynyl, 1-butynyl, 2-butynyl, and 3-butynyl. Alkynyl groups of other lengths are similarly branched or unbranched and substituted or unsubstituted.

By “C₂₋₆ heterocyclyl” is meant a stable 5- to 7-membered monocyclic or 7- to 14-membered bicyclic heterocyclic ring which is saturated, partially unsaturated, or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, carboxyl, PEG, alkyl-PEG, aryl-PEG, alkaryl-PEG, PEG-alkyl, PEG-aryl, and PEG-alkaryl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated it electrons (e.g., phenyl, biphenyl, napthyl, etc.). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, quaternary amino, PEG, alkyl-PEG, aryl-PEG, alkaryl-PEG, PEG-alkyl, PEG-aryl, and PEG-alkaryl groups. Aryl groups of other sizes are similarly substituted or unsubstituted.

By “C₇₋₁₄ alkaryl” is meant a C₁₋₄ alkyl substituted by a C₆₋₁₂ aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₁₀ heteroalkyl” is meant a branched or unbranched alkyl, alkenyl, or alkynyl group having from 1 to 10 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, carboxyl, PEG, alkyl-PEG, aryl-PEG, alkaryl-PEG, PEG-alkyl, PEG-aryl, and PEG-alkaryl groups. Examples of C₁₋₁₀ heteroalkyls include, without limitation, polyamines, methoxymethyl, and ethoxyethyl. Heteroalkyl groups of other lengths are similarly branched or unbranched and substituted or unsubstituted.

By “halide” is meant bromide, chloride, iodide, or fluoride.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

By “hydroxyalkyl” is meant a chemical moiety with the formula —(R)—OH, wherein R is selected from C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

By “alkoxy” is meant a chemical substituent of the formula -OR, wherein R is selected from C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, and C₁₋₁₀ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R″′)⁺, wherein R, R′, R″, and R″′ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

By “parent echinocandin class compound” is meant the echinocandin class compound which is modified by conjugation to a PEG, alkyl-PEG, aryl-PEG, alkaryl-PEG, PEG-alkyl, PEG-aryl, or PEG-alkaryl group to form an echinocandin class compound of the invention.

By “increased oral bioavailability” is meant the fraction of drug absorbed following oral administration to a subject is increased for the echinocandin class compound bearing a PEG, alkyl-PEG, aryl-PEG, alkaryl-PEG, PEG-alkyl, PEG-aryl, or PEG-alkaryl group in comparison to the parent echinocandin class compound (i.e., the echinocandin class compound lacking a PEG, alkyl-PEG, aryl-PEG, alkaryl-PEG, PEG-alkyl, PEG-aryl, or PEG-alkaryl group) orally administered under the same conditions (e.g., fasted or fed). The compounds of the invention can exhibit at least 25%, 50%, 100%, 200%, or 300% greater oral bioavailability than the corresponding parent echinocandin class compound from which they are derived.

By “increased transdermal bioavailability” is meant the fraction of drug absorbed following topical administration to a subject is increased for the echinocandin class compound bearing a PEG, alkyl-PEG, aryl-PEG, alkaryl-PEG, PEG-alkyl, PEG-aryl, or PEG-alkaryl group in comparison to the parent echinocandin class compound (i.e., the echinocandin class compound lacking a PEG, alkyl-PEG, aryl-PEG, alkaryl-PEG, PEG-alkyl, PEG-aryl, or PEG-alkaryl group) topically administered under the same conditions (e.g., with the same carriers and other inactive excipients). The compounds of the invention can exhibit at least 25%, 50%, 100%, 200%, or 300% greater transdermal bioavailability than the corresponding parent echinocandin class compound from which they are derived.

By “increased therapeutic index” is meant an increase in the ratio of median lethal dose (LD₅₀) to median effective dose (ED₅₀) for the echinocandin class compound bearing a PEG, alkyl-PEG, aryl-PEG, alkaryl-PEG, PEG-alkyl, PEG-aryl, or PEG-alkaryl group in comparison to the parent echinocandin class compound (i.e., the echinocandin class compound lacking a PEG, alkyl-PEG, aryl-PEG, alkaryl-PEG, PEG-alkyl, PEG-aryl, or PEG-alkaryl group) administered under the same conditions (e.g., with the same carriers and other inactive excipients and by the same route). The compounds of the invention can exhibit at least 25%, 50%, 100%, 200%, or 300% greater therapeutic index than the corresponding parent echinocandin class compound from which they are derived.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

As used herein, the terms “an amount sufficient” and “sufficient amount” refer to the amount of an echinocandin class compound required to treat or prevent an infection. The sufficient amount used to practice the invention for therapeutic or prophylactic treatment of conditions caused by or contributed to by an infection varies depending upon the manner of administration, the type of infection, the age, body weight, and general health of the subject. Ultimately, the attending physician or veterinarian will decide the appropriate amount and dosage regimen. Such amount is referred to as a “sufficient” amount.

The term “unit dosage form” refers to physically discrete units suitable as unitary dosages, such as a pill, tablet, caplet, hard capsule or soft capsule, each unit containing a predetermined quantity of an echinocandin class compound of the invention. By “hard capsule” is meant a capsule that includes a membrane that forms a two-part, capsule-shaped, container capable of carrying a solid or liquid payload of drug and excipients. By “soft capsule” is meant a capsule molded into a single container carrying a liquid or semisolid payload of drug and excipients.

By “fungal infection” is meant the invasion of a host by pathogenic fungi. For example, the infection may include the excessive growth of fungi that are normally present in or on the body of a subject or growth of fungi that are not normally present in or on a subject. More generally, a fungal infection can be any situation in which the presence of a fungal population(s) is damaging to a host body. Thus, a subject is “suffering” from a fungal infection when an excessive amount of a fungal population is present in or on the subject's body, or when the presence of a fungal population(s) is damaging the cells or other tissue of the subject.

Other features and advantages of the invention will be apparent from the following detailed description and the claims.

### DETAILED DESCRIPTION

The invention features echinocandin class compounds that have been modified to (i) have activity against one or more fungal species or genera; (ii) have increased aqueous solubility; (iii) have an increased therapeutic index; (iv) be suitable for topical administration; and/or (v) be suitable for oral administration. The echinocandin class compounds of the invention include, for example, a PEG, alkyl-PEG, aryl-PEG, alkaryl-PEG, PEG-alkyl, PEG-aryl, or PEG-alkaryl.

### Echinocandin Class Compounds

Echinocandin class compounds of the invention include compounds of any of formulas (I)-(V). These compounds can be synthesized, for example, as described in the examples by coupling functionalized or unfunctionalized echinocandin class compounds with the appropriate acyl, alkyl and/or amino groups under standard reaction conditions.

Typically, the semi-synthetic echinocandin class compounds of the invention are made by modifying the naturally occurring echinocandin scaffold. For example, pneumocandin B₀ is prepared by fermentation reactions; where fermentation and mixed broths produce a mixture of products which are then separated to produce pneumocandin B₀, which is used in the synthesis of caspofungin (see U.S. Pat. No. 6,610,822, which describes extraction of the echinocandin class compounds, such as, pneumocandin B₀, WF 11899 and echinocandin B by performing several extraction processes; and see U.S. Pat. No. 6,610,822, which describes methods for purifying the crude extracts).

For semi-synthetic approaches to echinocandin class compounds of the invention, the stereochemistry of the compound will be dictated by the starting material. Thus, the stereochemistry of the unnatural echinocandin derivatives will typically have the same stereochemistry as the naturally occurring echinocandin scaffold (representative examples are shown below for echinocandin B, anidulafungin, micafungin, and caspofungin) from which they are derived. Accordingly, any of the echinocandin class compounds shown below, echinocandin B, anidulafungin, micafungin, and caspofungin, can be used as a starting material in the synthesis of echinocandin class compounds of the invention which share the same stereochemical configuration at each of the amino acid residues found in the naturally occurring compound.

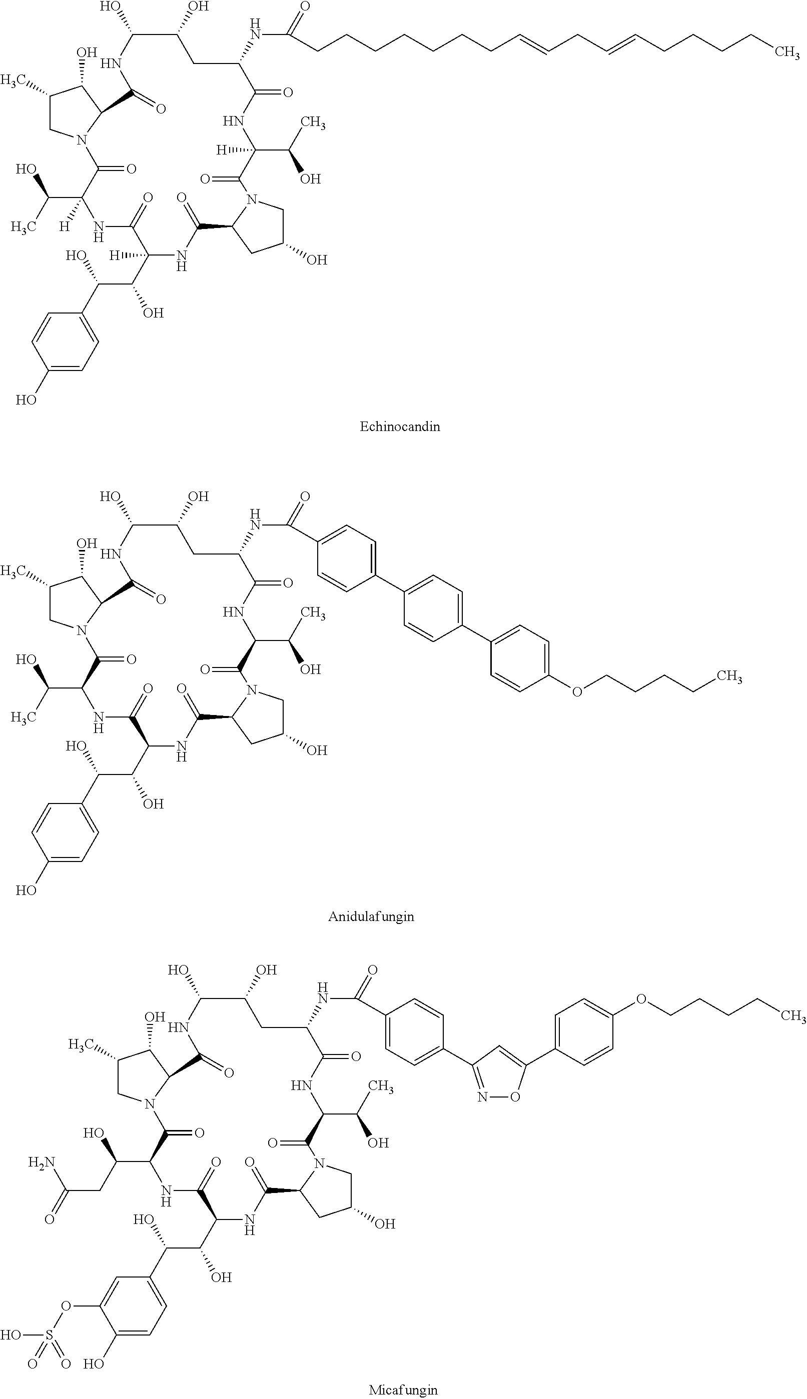

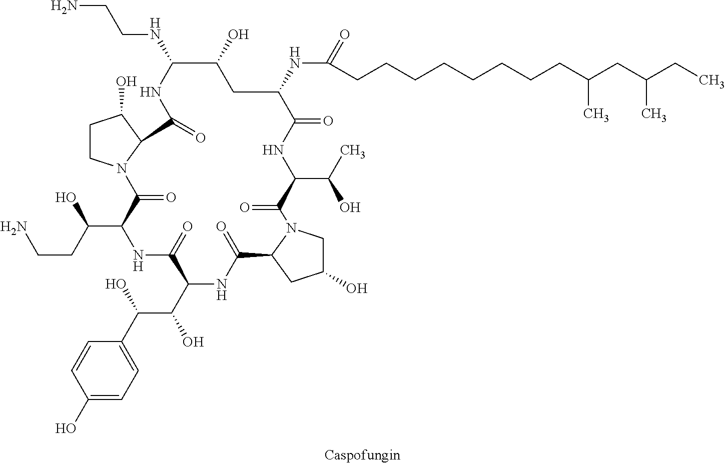

Accordingly, the echinocandin class compounds of the invention can be derived from the cyclic peptide antifungals which are produced by culturing various microorganisms. A number of cyclic peptide antifungals are known. Among these are echinocandin B (A30912A), aculeacin, mulundocandin, sporiofungin, L-671,329, FR901379, and S31794/Fl. All such antifungals are structurally characterized by a cyclic hexapeptide core, the amino group of one of the cyclic amino acids bearing a fatty acid acyl group forming a side chain off the core or nucleus. For example, echinocandin B has a linoleoyl side chain while aculeacin has a palmitoyl side chain. These fatty acid side chains of the cyclic hexa-peptides can be removed by enzymatic deacylation to provide a free amine terminus (e.g. R⁵ of formula (I) is H). Reacylation of the amino group of the nucleus provides semisynthetic antifungal compounds. For example, the echinocandin B core provides a number of antifungal agents when reacylated with certain unnatural side chain moieties (see Debono, U.S. Pat. No. 4,293,489). For example enzymatic deacylation of the echinocandin class compounds can be carried out with deacylase produced by the organism Actinoplanes utahensis and related microorganisms as described by Abbott et al., U.S. Pat. No. 4,293,482. Other synthetic modifications that can be made include derivatization of the aryl ring via a Mannich reaction (e.g. R⁴ of formula (I) is converted from H to CH₂NH₂); modification of the hydroxyl group at R¹ (e.g., R¹ of formula (I) is converted from OH to CH₂NH₂) (a sulfone or hemiaminal thioether intermediate is treated with NaCN to give the corresponding nitrile, and reduction of the nitrile affords the amino methyl derivative; see PCT Publication No. WO 96/22784 and Bansi Lal et al., Bioorganic and Medicinal Chemistry 11:5189 (2003)); alkylation of amine groups; amidation of amine groups; and the modifications described in the Examples.

The echinocandin class compounds can include substituents bearing branched PEG groups. Such branched PEGs can be prepared as described in Miller et al., Bioconjugate Chem., 17:267 (2006).

### Therapy and Formulation

The invention features compositions and methods for treating or preventing a disease or condition associated with a fungal infection by administering a compound of the invention. Compounds of the present invention may be administered by any appropriate route for treatment or prevention of a disease or condition associated with a fungal infection. These may be administered to humans, domestic pets, livestock, or other animals with a pharmaceutically acceptable diluent, carrier, or excipient. When administered orally, these may be in unit dosage form. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may he in the form of liquid solutions or suspensions; for oral administration, formulations may be in the form of tablets or capsules; and for intranasal formulations, in the form of powders, nasal drops, ear drops, or aerosols.

Methods well known in the art for making formulations are found, for example, in “Remington: The Science and Practice of Pharmacy” (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound or combination may be optionally administered as a pharmaceutically acceptable salt, such as non-toxic acid addition salts, alkali and alkaline earth salts (e.g., sodium, lithium, potassium, magnesium, or calcium salts), or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid, phosphoric acid, or the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc). Formulations for oral use may also be provided in unit dosage form as chewable tablets, tablets, caplets, or capsules (i.e., as hard gelatin capsules wherein the active ingredient is mixed with an inert solid diluent, or as soft gelatin capsules wherein the active ingredient is mixed with water or an oil medium).

The formulations can be administered to human subjects in therapeutically effective amounts. Typical dose ranges are from about 0.01 ug/kg to about 800 mg/kg, or about 0.1 mg/kg to about 50 mg/kg, of body weight per day. The preferred dosage of drug to be administered is likely to depend on such variables as the type and extent of the disorder, the overall health status of the particular subject, the specific compound being administered, the excipients used to formulate the compound, and its route of administration.

The compounds of the invention can be used to treat, for example, tinea capitis, tinea corporis, tinea pedis, onychomycosis, perionychomycosis, pityriasis versicolor, oral thrush, vaginal candidosis, respiratory tract candidosis, biliary candidosis, eosophageal candidosis, urinary tract candidosis, systemic candidosis, mucocutaneous candidosis, aspergillosis, mucormycosis, paracoccidioidomycosis, North American blastomycosis, histoplasmosis, coccidioidomycosis, and sporotrichosis.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

Analytical HPLC was performed using the following column(s) and conditions: Phenomenex Luna C18(2), 5 μm, 100 Å, 2.0×150 mm, 1-99% CH₃CN (0.1% TFA) in H₂O (0.1% TFA)/15 min. Preparative HPLC was performed using the following column: Waters Nova-Pak HR C 18, 6 μm, 60 Å, 19×300 mm, CH₃CN/H₂O various linear gradients and modifiers as necessary at 10 mL/min.

The following abbreviations are used in the examples below: min (minutes), hr (hours), mmol (millimole), mL (milliliter), μm (micron), A (angstrom), THF (tetrahydrofuran), DMF (dimethylformamide), TLC (thin layer chromatography), TFA (trifluoroacetic acid), HPLC (high performance liquid chromatography), RP (reversed phase), DIEA (diisopropylethylamine), LC/MS (liquid chromatography/mass spectrometry), MEG (methyl ethylene glycol), MPEG (methyl polyethyleneglycol), T_{R} (retention time on HPLC), C (Celsius), and FMOC (fluorenylmethyloxycarbonyl).

### Example 1

### Synthesis of Compound MPEG₇Hexyl Alcohol

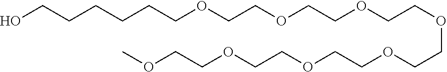

MPEG₇hexyl succinimidyl ester (650 mg; 1.16 mmol) in 5 ml of diethyl ether under argon atmosphere was treated with LiBH₄ (59 mg; 2.71 mmol). The mixture was stirred at room temp overnight. Solvent had evaporated and the mixture was reconstituted in dry THF. TLC [SiO₂; 10% methanol/dichloromethane; KMnO₄ detection] showed remaining starting material, and the reaction was treated with additional LiBH₄ (approx. 50 mg; 2.3 mmol). The mixture was monitored by TLC and heated to reflux for approx. 4 hr until no starting material remained, and a single product was observed. The reaction was quenched by slow addition of saturated aqueous NaHCO₃. Approx. 25 mL of dichloromethane was added and the mixture was separated. The organic phase was washed once each with approx. 25 ml of aq. NaHCO₃ and brine then dried with MgSO₄ and conc. in vacuo to give 441 mg (85% TY) of MPEG hexyl alcohol as a clear colorless oil.

### Example 2

### Synthesis of Compounds 1 and 2

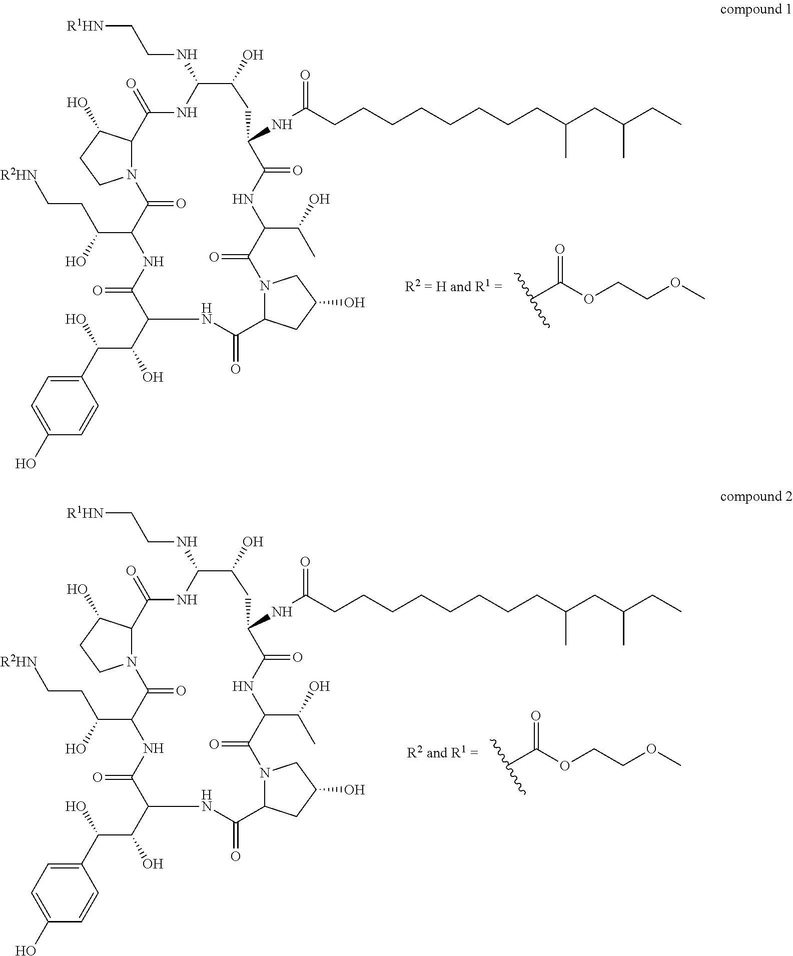

Caspofungin acetate (10 mg; 0.008 mmol) in 0.5 mL THF was treated with 2-methoxyethanol-succinimidyl carbonate (175 μL, at 10 mg/mL in THF; 1.75 mg; 0.008 mmol). The resulting solution was stirred at ambient temperature for ca. 45 minutes and developed two major products and one minor product. The solution was concentrated in vacuo at room temperature, diluted with water, and the two major products were separated by preparative RP HPLC eluting with 0.05M CH₃CO₂⁻NH₄⁺ (pH 5.0)/CH₃CN. The purified products were isolated by freeze-drying to give white solids. The reaction was repeated until sufficient quantities of purified samples were accumulated, and the products were combined based on purity to give 7.9 mg of monoconjugate, compound 1, and 5.7 mg of diconjugate, compound 2. HPLC T_{R} 9.92 min (84%); LC/MS, ESI+, m/z 598.3 [M+2H]⁺. HPLC T_{R} 11.04 min (97%); LC/MS, ESI+, m/z 1297.7 [M+H]⁺, 660.3 [M+H+Na]²⁺

### Example 3

### Synthesis of Compounds 3 and 4

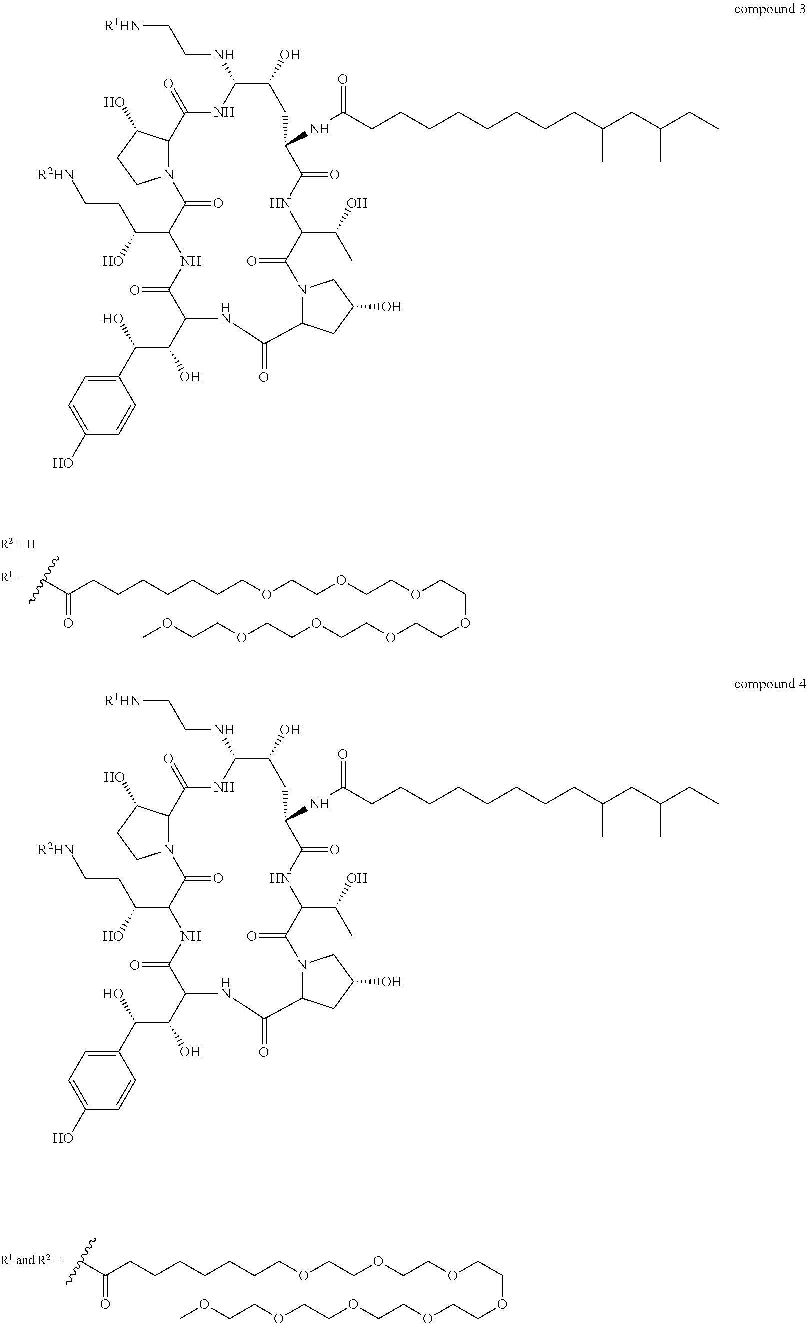

Caspofungin diacetate (15 mg; 0.012 mmol) dissolved in 0.5 mL THF and 4 drops of water was treated with MPEG₇octyl succinimidyl ester (7.2 mg; 0.012 mmol). The resulting solution was stirred at ambient temperature for approximately 15 hr then concentrated in vacuo at room temperature and purified by preparative RP HPLC eluting with water (0.1% TFA)/CH₃CN (0.1% TFA). Pure fractions of interest were freeze dried to provide 7.3 mg of monoconjugate, compound 3, and 4.7 mg of diconjugate, compound 4, as white solids: HPLC T_{R} 10.38 min (95.2%); LC/MS, ESI+m/z 1557.9 [M+H]⁺, 779.5 [M+2H]²⁺, 527.3 [M+2H+Na]^{3±}. HPLC T_{R} 11.71 min (97%); LC/MS, ESI+m/z 1023 [M+H+Na]²⁺, 1034 [M+2Na]^{2±}, 689.7 [M+H+2Na]³⁺.

### Example 4

### Synthesis of Compound 5

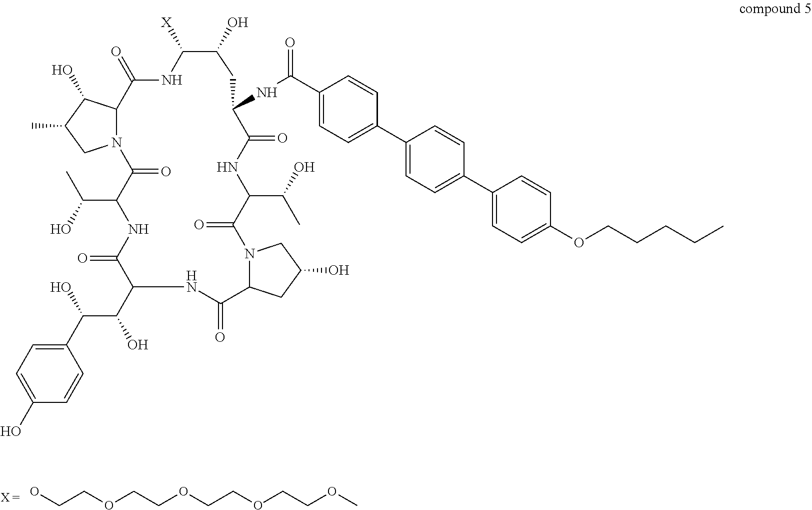

Anidulafungin (10 mg; 0.008 mmol) in 1,4-dioxane was treated with MPEG₄OH (78 mg; 0.37 mmol) added neat. p-Toluenesulfonic acid was added (0.020 ml at 2.8 mg/ml in 1,4-dioxane; 0.06 mg; 0.0003 mmol) and the resulting clear solution was stirred at ambient temperature for 3 days. The solution was diluted with water and acetonitrile and purified by preparative RP HPLC eluting with water and acetonitrile. Product was isolated by freeze-drying to give 5.1 mg of compound 5 as a white solid. HPLC T_{R} 12.32 min (90.3%). LC/MS, ESI+, m/z 1352.6 [M+Na]⁺.

### Example 5

### Synthesis of Compound 6

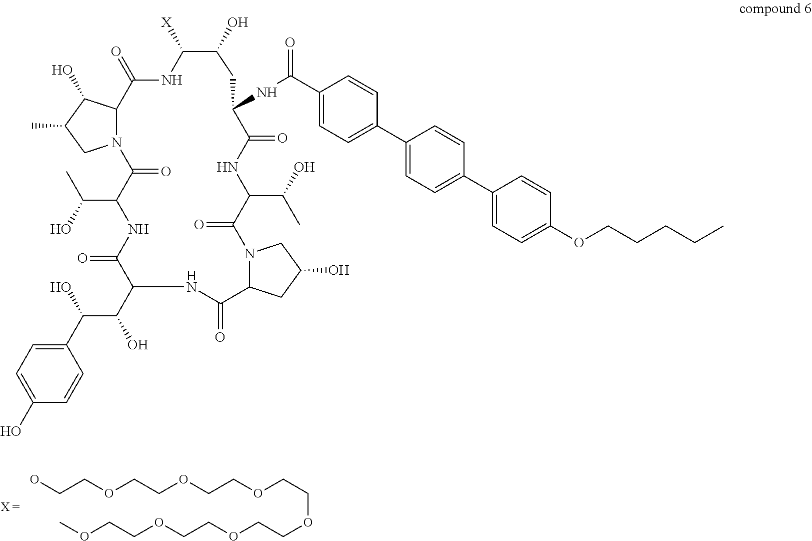

Anidulafungin (10 mg; 0.008 mmol) was added to MPEG₇OH (156 mg; 0.46 mmol) to give a viscous solution. p-Toluenesulfonic acid (0.030 ml at 2.8 mg/ml in 1,4-dioxane; 0.08 mg; 0.0004 mmol) was added, and the resulting solution was stirred at ambient temperature for 2 days. The solution was diluted with water and acetonitrile and purified by preparative RP HPLC eluting with water and acetonitrile. Product was isolated by freeze-drying to give 4.5 mg of compound 6 as a white solid. HPLC T_{R} 12.21 min (92.2%); LC/MS, ESI+m/z 1484.7 [M+Na]⁺, 753.8 [M+2Na]²⁺.

### Example 6

### Synthesis of Compound 7

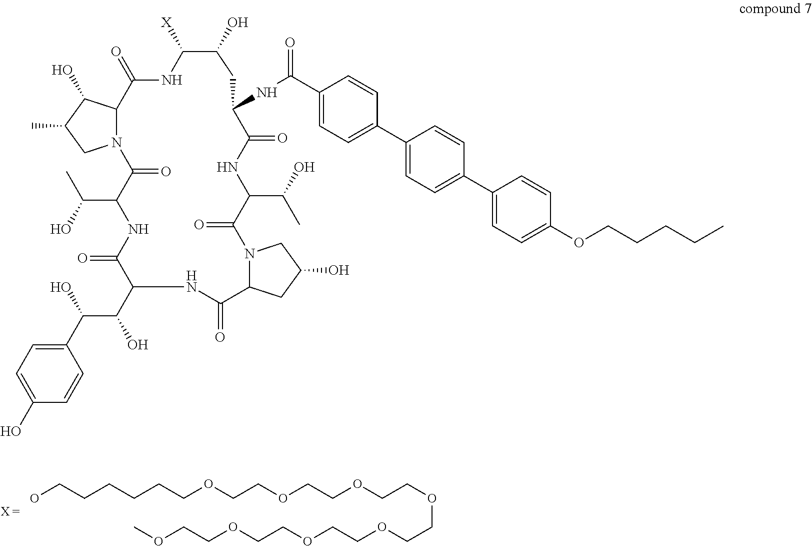

Anidulafungin (11 mg; 0.010 mmol) was mixed with MPEG₇C₆OH (56 mg; 0.13 mmol). Dry DMF (10 drops) was added to give a clear solution which was treated with HCl (4M in 1,4-dioxane; 2.5 μL; 0.01 mmol). The solution was stirred at ambient temperature for 6 hr then at 4° C. for approximately 15 hr. The solution was diluted with water and acetonitrile and purified by preparative RP HPLC eluting with water and acetonitrile. Product was isolated by freeze-drying to give 7.2 mg of compound 7 as a white solid. HPLC T_{R} 12.72 min (84.5%). LC/MS, ESI+, m/z 1584.8 [M+Na]⁺, 1562.8 [M+H]⁺.

### Example 7

### Synthesis of Compound 8

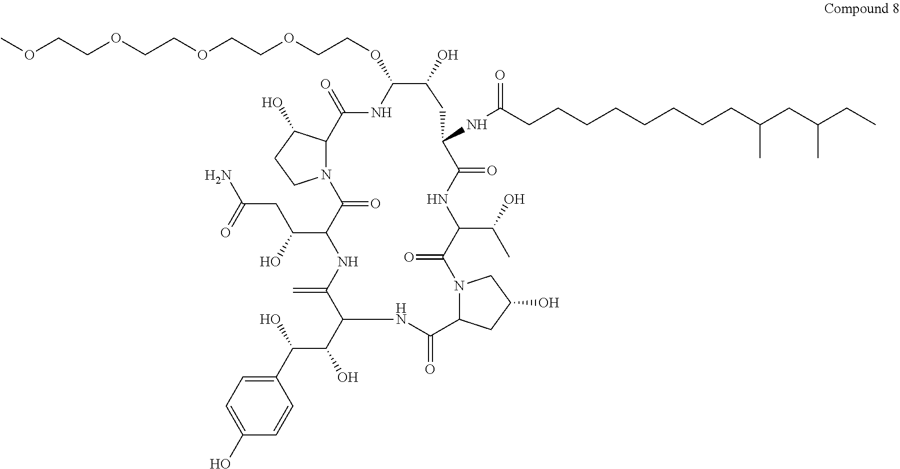

Pneumocandin B₀ (11 mg; 0.010 mmol) was suspended in MPEG₄OH (100 mg; 0.48 mmol). Dry DMF (5 drops) was added to give a clear solution which was treated with HCl (4M in 1,4-dioxane; 2.5 μL; 0.01 mmol). The solution was stirred for 3 hr then diluted with water and acetonitrile and purified by preparative RP HPLC eluting with water and acetonitrile. Product was isolated by freeze-drying to give 6.5 mg of compound 9 as a white solid. HPLC T_{R} 11.76 min (96.9%). LC/MS, ESI+, m/z 1277.7 [M+Na]⁺.

### Example 8

### Synthesis of Compound 9

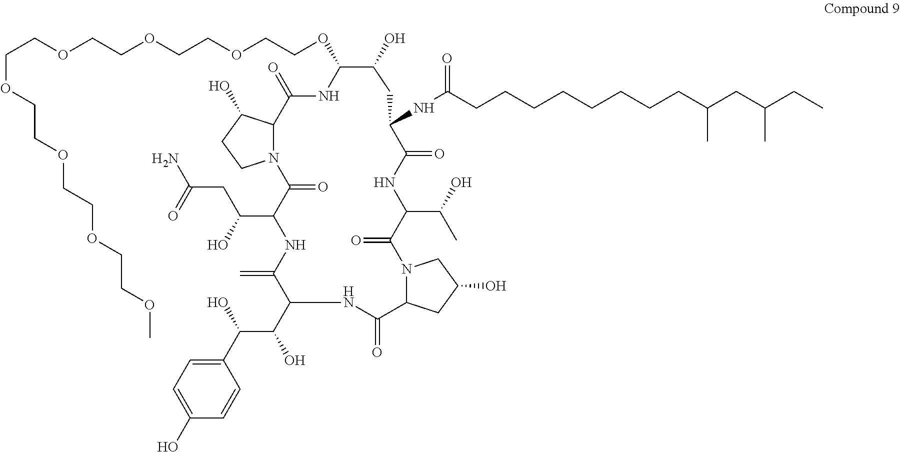

Pneumocandin B₀ (10 mg; 0.009 mmol) was suspended in MPEG₇OH (135 mg; 0.40 mmol). The mixture was treated with HCl (4M in 1,4-dioxane; 2.5 μL; 0.01 mmol) and a few drops of dry DMF were added. The solution was stirred overnight then diluted with water and acetonitrile and purified by preparative RP HPLC eluting with water and acetonitrile. Product was isolated by freeze-drying to give 4.4 mg of compound 10 as a white solid. HPLC T_{R} 11.62 min (97.8%). LC/MS, ESI+, m/z 1409.8 [M+Na]⁺, 1387.8 [M+H]⁺.

### Example 9

### Synthesis of Compound 10

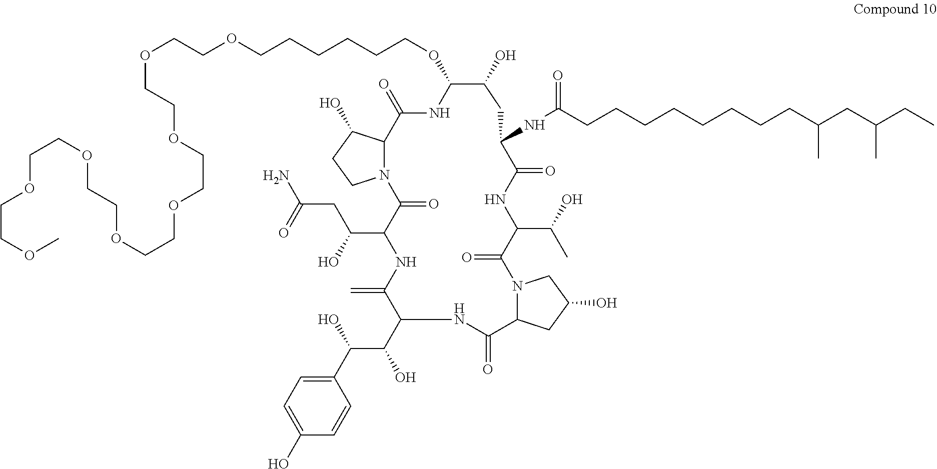

Pneumocandin B₀ (12.5 mg; 0.012 mmol) was suspended in MPEG₇C₆OH (65 mg; 0.15 mmol). Dry DMF (5 drops) was added and the mixture was stirred and sonicated to give a clear solution which was treated with HCl (4M in 1,4-dioxane; 2.5 μL; 0.01 mmol). The solution was stirred at ambient temperature for 18 hr then diluted with water and acetonitrile and purified by preparative RP HPLC eluting with water and acetonitrile. Product was isolated by freeze-drying to give 6.7 mg of compound 10 as a white solid. HPLC T_{R} 12.09 min (96.3%). LC/MS, ESI+, m/z 1509.8 [M+Na]⁺, 1487.9 [M+H]⁺.

### Example 10

### In vitro Activity of Echinocandin Class Compounds

MIC values (μg/mL) of echinocandin class compounds of the invention against various Candida and Aspergillus species were obtained as follows. Test organisms were obtained from the American Type Culture Collection (Manassas, Va.). The isolates were maintained at −80 ° C., then thawed and sub-cultured one day prior to testing (seven days for Aspergillus strains).

The test media for the MIC assays against yeast and filamentous fungi was as follows: RPMI-1640 (buffered with 0.165M MOPS, pH 7.0) was prepared at 105% to offset the presence of 5% drug by volume when no mouse serum was present and 155% to offset the presence of the drug at 5% and mouse serum. The mouse serum was heat-inactivated for 30 minutes at 56° C., and filtered through a 0.2 μm low protein binding filter unit prior to use.

MIC values were determined using broth microdilution. The wells of the daughter plates ultimately contained 180 μL of RPMI-1640 medium with and without mouse serum, 10 μL of drug solution, and 10 μL of yeast or fungal inoculum.

Prior to susceptibility testing, fungal isolates were removed from frozen storage, thawed at room temperature, and subcultured to Potato Dextrose Agar. The Candida strains were incubated overnight at 35° C. and the Aspergillus strains for 7 days at 25° C. Yeast colonies were inoculated into sterile saline and adjusted to the turbidity of a 0.5 McFarland standard. Inoculums from Aspergillus strains were prepared by washing the surface of an agar slant with 1 mL of sterile saline containing 0.05% of Tween 20. The resulting colloidial suspensions were adjusted to an optical density of 0.09 to 0.11 at λ 625 nm.

Both the yeast and filamentous fungus suspensions were further diluted 1:100 in RPMI-1640 medium and dispensed into sterile reservoirs and used to inoculate the daughter plates. 10 μL of standardized inoculum was delivered into each well. This yielded a final cell concentration in the daughter plates of approximately 2.5×10³ colony-forming units/mL. Plates were incubated at 35° C. for approximately 48 hr.

Following incubation, microplates were viewed from the bottom using a plate viewer. An un-inoculated solubility control plate was observed for evidence of drug precipitation. The MIC was defined as the lowest concentration of an antifungal agent that substantially inhibits growth of the organism as detected visually. MIC determinations were done at 48 hr for Candida strains. For the Aspergillus strains, the growth in each MIC well was compared with that of the growth control at 48 hr.

TABLE 1

| | C. albicans | | C. parapsilosis | | A. flavus1 | |
|---|---|---|---|---|---|---|
| | 104 | | 630 | | 626 | |
| | ATCC 90028 | | ATCC 90018 | | ATCC 64025 | |
| Echinocandin | 0% | 50% | 0% | 50% | 0% | 50% |
| Compound | Serum | Serum | Serum | Serum | Serum | Serum |
| Anidulafungin | 0.03 | 0.06 | 2 | >16 | <0.015 | 0.03 |
| 5 | 2 | 2 | >16 | >16 | 0.125 | 0.5 |
| 6 | 2 | 8 | >16 | >16 | 0.125 | 0.5 |
| 7 | 2 | >16 | >16 | >16 | 0.25 | 2 |
| Caspofungin | 0.125 | 0.06 | 0.5 | 2 | 0.06 | NA |
| 1 | 0.5 | 1 | 2 | >16 | 0.125 | 0.5 |
| 2 | 4 | >16 | 16 | >16 | NA | 4 |
| 3 | 2 | >16 | >16 | >16 | 2 | 8 |
| 4 | 8 | >16 | >16 | >16 | 16 | NA |
| Pneumocandin | 0.25 | 4 | 8 | >16 | 1 | 0.25 |
| B₀ | | | | | | |
| 8 | 8 | >16 | >16 | >16 | 0.5 | 2 |
| 9 | >16 | >16 | >16 | >16 | 2 | 8 |
| 10 | >16 | >16 | >16 | >16 | 4 | 16 |

1Growth reduction of 50%
NA: Not available

### Example 11

### A 1:1 Mixture of Compounds 3 and 11

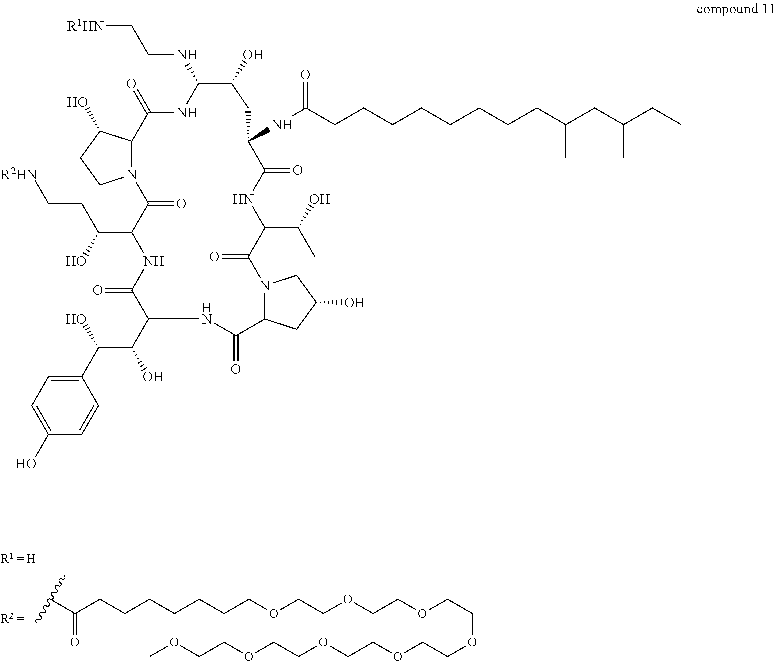

Isolated from preparations of compound 3, enriched monoconjugate samples were combined to give a 1:1 mixture of monoconjugates (i.e., a 1:1 mixture of compounds 3 and 11). LC/MS: 2 components by UV detection in a ratio of 100:97, ESI+m/z 1557.95 [M+H]⁺ for both components.

### Example 12

### Synthesis of Compound 12

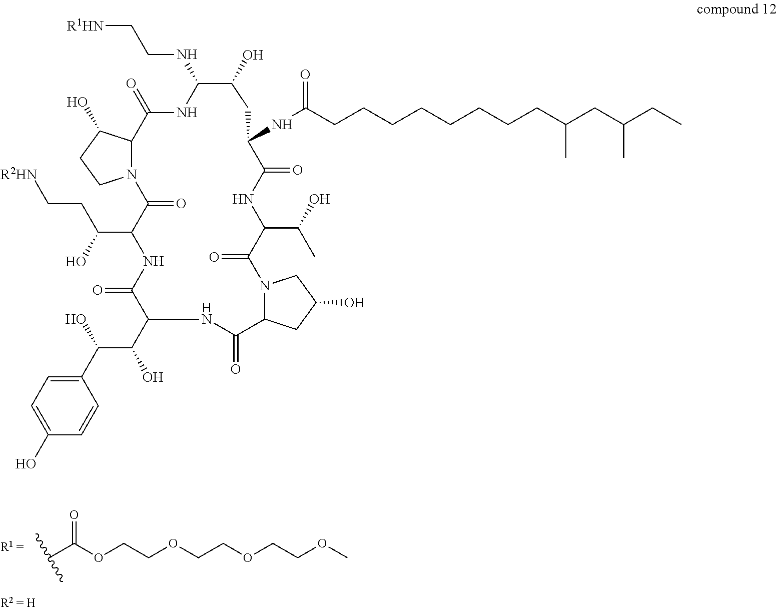

Caspofungin acetate (5 mg; 0.004 mmol) in 0.25 mL THF and 3 drops of water was treated with MPEG3-succinimidyl carbonate (1.2 mg; 0.004 mmol). The resulting solution was stirred at ambient temperature overnight. The solution was concentrated under an air stream and then diluted with water and acetonitrile and purified by preparative RP HPLC eluting with water (0.1% TFA)/CH₃CN (0.1% TFA). The reaction was repeated to give sufficient product, and the purified product was isolated by freeze-drying and combined to give 3.5 mg of compound 12 as white solid. LC/MS, 85% purity by LC; ESI+, m/z 1283.73 [M+H]⁺.

### Example 13

### Synthesis of Compound 13

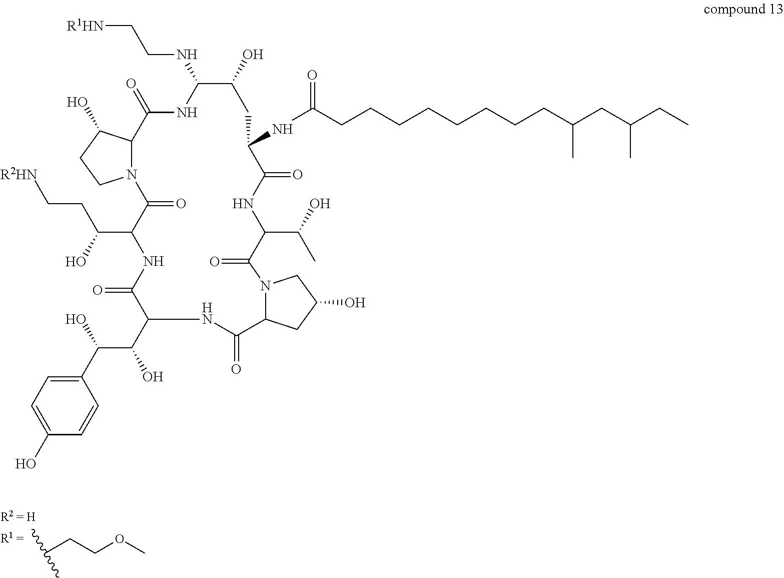

Caspofungin acetate (5 mg; 0.004 mmol) in 0.2 mL DMF was treated with DIEA (3.0 μL; 0.017 mmol) and 1-bromo-2-methoxyethane (1.5 μL; 0.016 mmol). The resulting solution was heated at 35-40° C. for 3 days. The reaction was diluted with water and purified by preparative RP HPLC eluting with water (0.1% TFA)/CH₃CN (0.1% TFA). The reaction was repeated to give sufficient product, and the purified product was isolated by freeze-drying and combined to give 2.1 mg of compound 13 as white solid. HPLC T_{R} 9.54 min (94%); LC/MS, ESI+/−m/z 1551.71 [M+H]+, 1149.67 [M−H]⁻, 1033.59 [imine fragment from aminal elimination]+(calculated 1033.58) confirms R¹ substitution.

### Example 14

### Synthesis of Compound 14

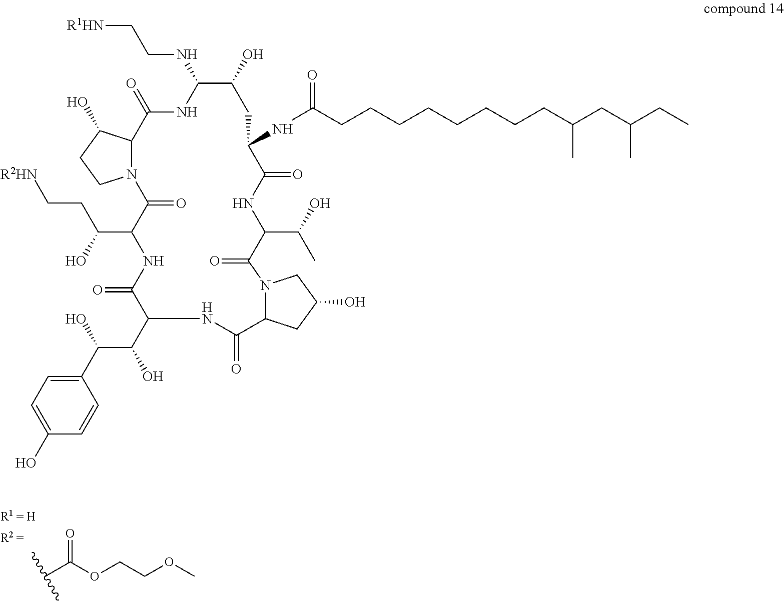

Caspofungin acetate (4.9 mg; 0.004 mmol) in anhydrous DMF was treated with FMOC- succinimidyl carbonate (1.4 mg; 0.004 mmol). The solution was stirred at 4° C. for 3.5 hr. The reaction was then treated with 2-methoxyethanol-succinimidyl carbonate (3 mg; 0.014 mmol) and allowed to stir overnight at room temperature. The reaction was then treated with 5% piperidine in DMF. HPLC analysis showed no remaining FMOC protected intermediates. The mixture was diluted with water and acidified by titration with TFA to obtain a clear solution which was purified by preparative RP HPLC eluting with water (0.1% TFA)/CH₃CN (0.1% TFA). The reaction was repeated to give sufficient product. Product was isolated by freeze-drying and combined to give 2.6 mg of compound 14 as white solid. HPLC TR10.06 min (85%); LC/MS, ESI+/−m/z 1195.68 [M+H]⁺, 1193.67 [M−H]⁻. No imine fragment observed in positive ion mode.

### Example 15

### Synthesis of Compound 15

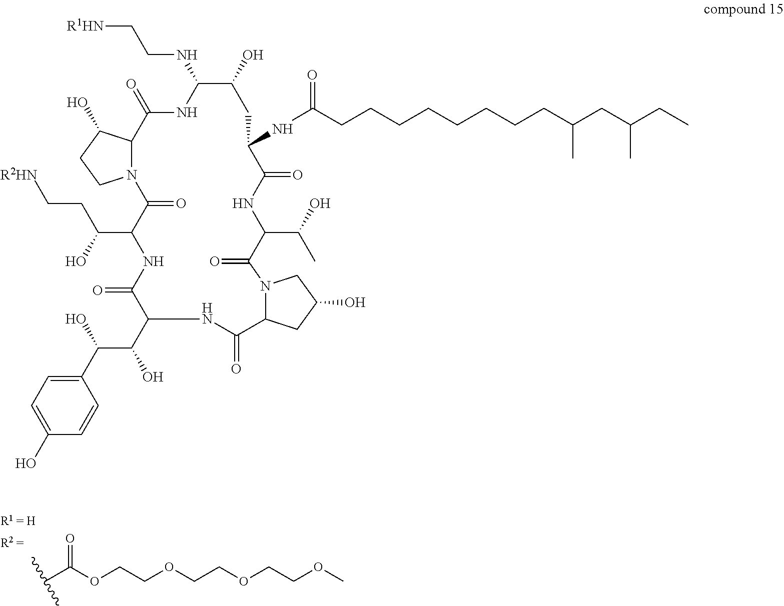

Caspofungin acetate (10 mg; 0.008 mmol) in anhydrous DMF was treated with FMOC-succinimidyl carbonate (2.7 mg; 0.008 mmol). The solution was stirred at 4° C. until the starting material was consumed then treated with MPEG3-succinimidyl carbonate (3 mg; 0.010 mmol) and allowed to stir overnight at room temperature. The reaction was then treated with 5% piperidine in DMF. HPLC analysis showed no remaining FMOC protected intermediates. The mixture was diluted with water and acetonitrile and acidified by titration with TFA to obtain a clear solution which was purified by preparative RP HPLC eluting with water (0.1% TFA)/CH₃CN (0.1% TFA). Product was isolated by freeze-drying to give 3.0 mg of compound 15 as white solid. HPLC T_{R} 10.12 min (85%); LC/MS, ESI+m/z 1283.73 [M+H]⁺. No imine fragment observed in positive ion mode.

### Example 16

### Synthesis of Compound 16

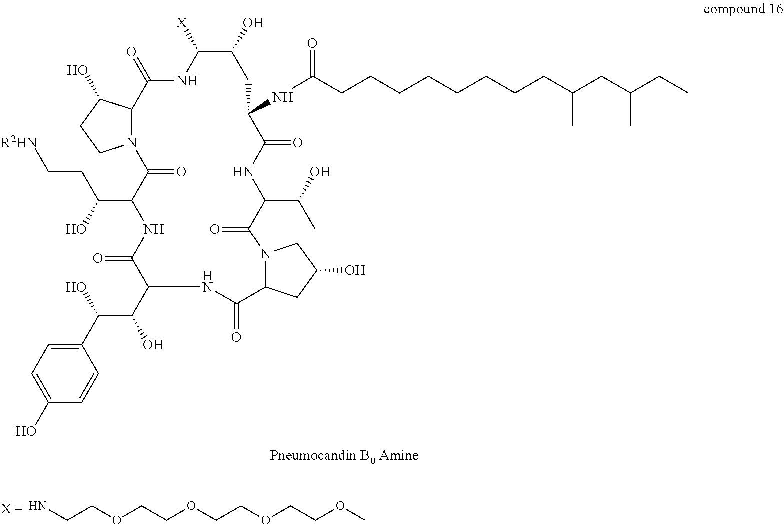

Pneumocandin hemiaminal-(4-methoxy)phenylthioether: Pneumocandin B₀ (103 mg; 0.097 mmol) suspended in acetonitrile was cooled to −15° C. and treated with 4-methoxythiophenol (13 μL; 0.107 mmol) followed by TFA (0.28 mL). The resulting mixture was stirred at −15 to −20° C. overnight then quenched by slow addition of water (5 mL). The resulting suspension was stirred at 0° C. for 30 min then separated. The precipitate was twice re-suspended in 25% acetonitrile/water (5 mL) with sonication and stirring then separated. The solids were then dried in vacuo overnight to give 94 mg of pneumocandin hemiaminal-(4-methoxy)phenylthioether as a white solid. HPLC T_{R} 12.75 min (78%). LC/MS, ESI+/−m/z 1187.60 [M+H]⁺, 1185.58 [M−H]⁻.

Pneumocandin hemiaminal-(4-methoxy)phenylthioether amine: Under anhydrous conditions and argon atmosphere, pneumocandin hemiaminal-(4-methoxy)phenylthioether (93 mg; <0.08 mmol) was suspended in THF (4 mL) and treated with phenylboronic acid (13 mg; 0.11 mmol) followed by activated 3 Å molecular sieves. The mixture was stirred overnight at room temperature to give a clear solution that was concentrated to ˜2 mL under positive argon flow. The solution was treated with additional molecular sieves and stirred for 30 min then transferred to a dry flask. The molecular sieves were rinsed with dry THF which was also transferred to give a total volume of 4 mL. The reaction was further diluted with 0.5 mL of dry 1,4-dioxane then cooled to −10° C. and treated with BH₃-dimethyl sulfide (2.0M in THF; 250 μL; 0.50 mmol). After 3 hr, the solution formed a gel and was further diluted with 1,4-dioxane and treated with additional BH3-dimethyl sulfide (80 μL; 0.16 mmol). The reaction was stirred for 2 hr longer at −10° C. then quenched by slow addition of 1.0M HCl (0.20 mL). The resulting solution was concentrated to ca. 2 mL then diluted with water to ca. 10 mL and purified by preparative RP HPLC eluting with water (0.1% TFA)/CH₃CN (0.1% TFA). Product was isolated by freeze-drying to give 36 mg of the title compound as a white solid and presumed TFA salt. HPLC T_{R} 11.69 min (>99%). LC/MS, ESI+/−m/z 1173.62 [M+H]⁺, 1207.58 [M+Cl]⁻.

Compound 16: Pneumocandin hemiaminal-(4-methoxy)phenylthioether amine (10 mg; 0.008 mmol), MPEG₄NH₂ (0.1 mL; ˜0.5 mmol), and DIEA (20 ESL; 0.12 mmol) were mixed and heated at 60° C. overnight. The resulting solution was diluted with methanol (0.5 mL) and water (1 mL) and acidified with acetic acid then purified by preparative RP HPLC eluting with water (0.1% TFA)/CH₃CN (0.1% TFA). Product was isolated by freeze-drying to give 6.4 mg of compound 16 as a white solid. HPLC T_{R} 10.72 min (94%). LC/MS, ESI+/−m/z 1240.73 [M+H]+, 1238.72 [M−H]⁻.

### Example 17

### Synthesis of Compound 17

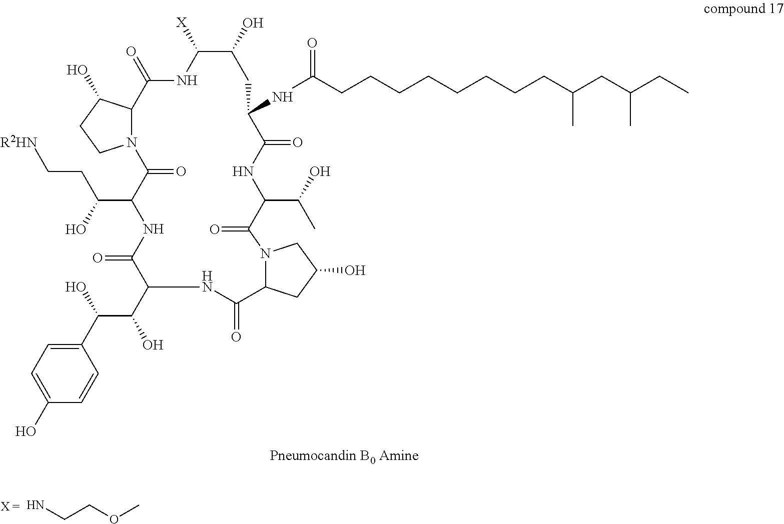

Pneumocandin hemiaminal-(4-methoxy)phenylthioether amine (20 mg; 0.016 mmol) was dissolved in 2-methoxyethylamine (0.1 mL). The solution was heated at 40° C. overnight then at 60° C. for 4 hr then diluted with methanol (0.5 mL) and water (2 mL) and acidified with TFA. The acidified mixture was further diluted with water and methanol then purified by preparative RP HPLC eluting with water (0.1% TFA)/CH₃CN (0.1% TFA). Product was isolated by freeze-drying to give 12 mg of Compound 17 as a white solid. HPLC T_{R} 9.91 min (97%). LC/MS, ESI+/−m/z 1108.65 [M+H]⁺, 1106.64 [M−H]⁻.

### Example 18

### Synthesis of Compound 18

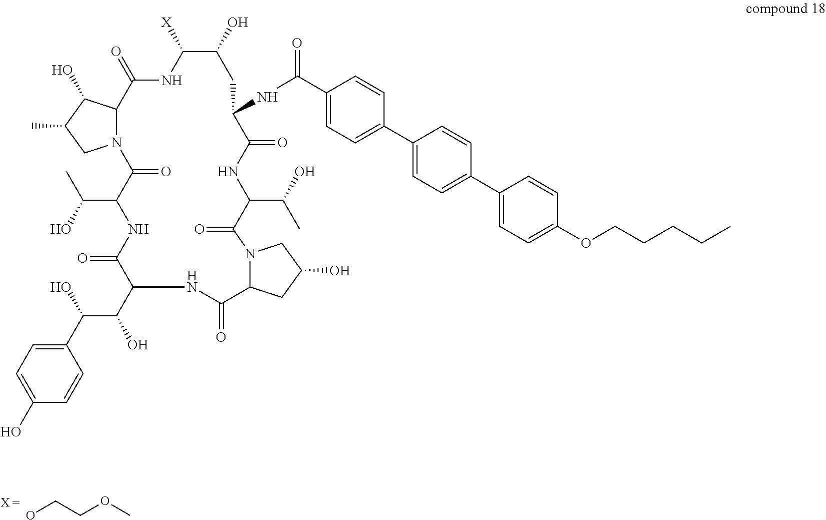

Anidulafungin (4 mg; 0.003 mmol) dissolved in 2-methoxy ethanol (˜200 mg) was treated with HCl (4M in 1,4-dioxane; 0.5μL; 0.002 mmol). The resulting solution was stirred at room temperature for 30 min then diluted with ˜120 volumes of water. Precipitated solids were separated and purified by preparative RP HPLC eluting with water and acetonitrile. The reaction was repeated to give sufficient product which was isolated by freeze-drying and combined to give 4.3 mg of compound 18 as a white solid. HPLC T_{R} 12.34 min (98.9%). LC/MS, ESI+m/z 1220.5 [M+Na]⁺, 1198.5 [M+H]⁺.

### Example 19

### Synthesis of Compound 19

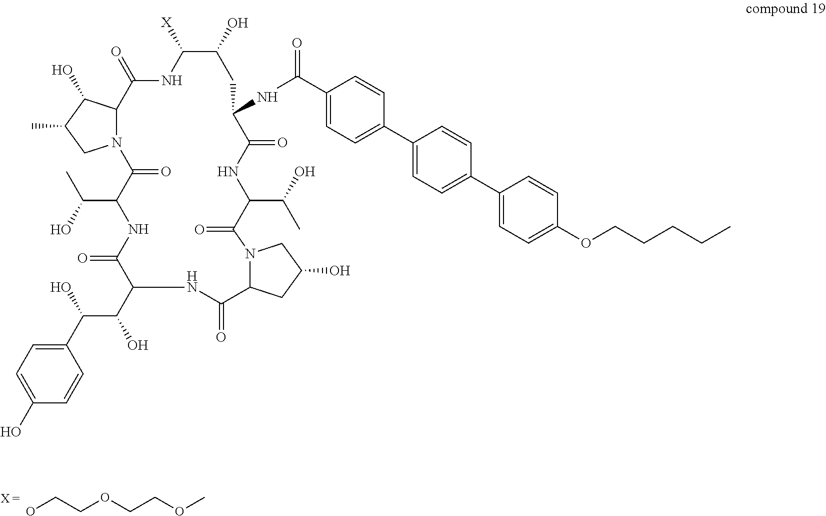

Anidulafungin (5.5 mg; 0.0048 mmol) dissolved in MPEG2OH (˜200 mg) was treated with HCl (4M in 1,4-dioxane; 1.0 μL; 0.004 mmol). The resulting solution was stirred at room temperature for 1 hr then diluted with ˜120 volumes of water. Precipitated solids were separated and purified by preparative RP HPLC eluting with water and acetonitrile. The product was isolated by freeze-drying to give 2.2 mg of compound 19 as a white solid. HPLC T_{R} 12.34 min (>99%). LC/MS, ESI+m/z 1264.5 [M+Na]⁺.

### Example 20

### Synthesis of Compound 20

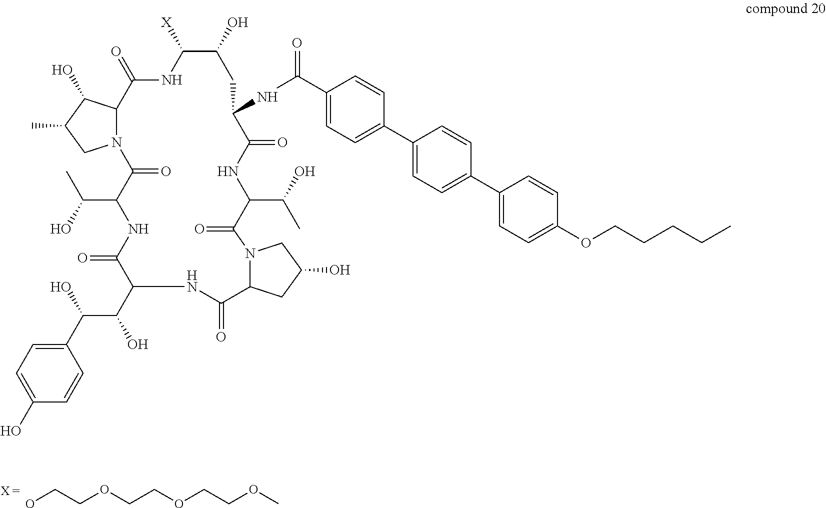

Anidulafungin (6.3 mg; 0.0055 mmol) dissolved in MPEG3OH (˜200 mg) was treated with HCl (4M in 1,4-dioxane; 1.0 μL; 0.004 mmol). The resulting solution was stirred at room temperature for lhr then diluted with ˜120 volumes of water. Precipitated solids were separated and purified by preparative RP HPLC eluting with water and acetonitrile. The product was isolated by freeze-drying to give 4.0 mg of compound 20 as a white solid. HPLC T_{R} 12.36 min (>98%). LC/MS, ESI+m/z 1308.5 [M+Na]⁺, 1286.6 [M+H]⁺.

### Example 21

### Synthesis of Compound 21

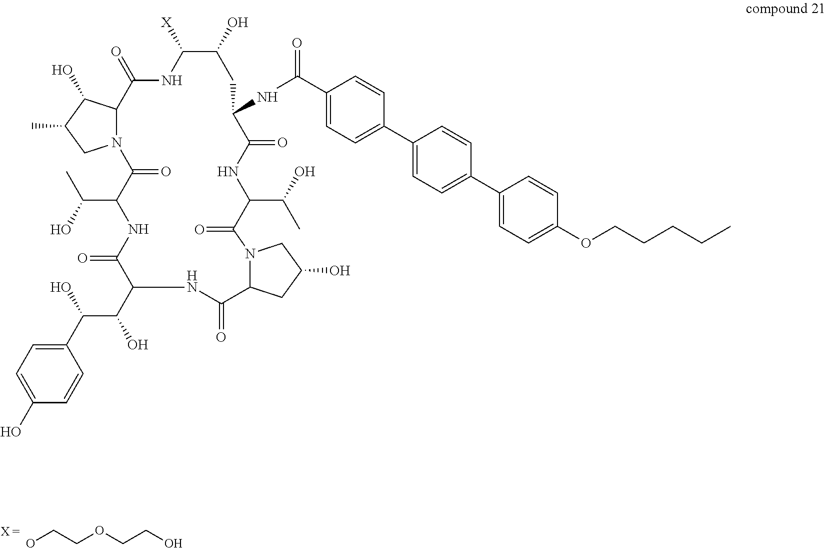

Anidulafungin (20 mg; 0.018 mmol) dissolved in diethylene glycol (0.2 mL) was treated with HCl (4M in 1,4-dioxane; 4.0 μL; 0.016 mmol). The resulting solution was stirred at room temperature for 2 days then at −20° C. for 3 days. The solution was diluted with water and methanol and purified by preparative RP HPLC eluting with water and acetonitrile. The product was isolated by freeze-drying to give 6.6 mg of compound 21 as a white solid. HPLC T_{R} 11.83 min (>99%). LC/MS, ESI+/−m/z 1228.6 [M+H]⁺, 1210.55 [M−OH]⁺, 1226.56 [M−H]⁻.

### Example 22

### In vivo Activity of Echinocandin Class Compounds

The objective of these studies was to evaluate the efficacy of test compounds in the mouse candidiasis infection model.

Inoculum Preparation

The strain, C. albicans R303 was transferred from frozen storage onto Sabauroud dextrose agar (SDA) plates and grown for ˜24 hr at 35° C. The inoculum was prepared by transferring colonies from the plate to phosphate buffered saline (PBS) and the concentration adjusted to 10⁶ CFU/mL with the aid of a spectrophotometer. The stock was diluted 1:9 to prepare the inoculum. Prior to each run the concentration was verified using the dilution plate count method.

The female CD-1 mice used in this study were obtained from Charles River Laboratories, Portage, MI. The animals were approximately seven-weeks-old at the start of the study and weighed about 16-26 g.

In one approach, mice were made neutropenic with IP injections of cyclophosphamide (150 mg/kg in 10 mL/kg) at 4 and 1 day before inoculation. Each animal was inoculated with the appropriate concentration by injecting 0.1 mL of inoculum into a tail vein. The test compounds were administered IP at 2 hr after infection.

The kidneys were collected from four mice in control group 1 (untreated) at 2 hr after infection, and from the remaining mice in the study at 24 hr after infection. Kidneys were removed aseptically from each mouse and were combined in a sterile tube. An aliquot (2 mL) of sterile PBS was added to each tube and the contents homogenized with a tissue homogenizer (Polytron 3100). Serial dilutions of the tissue homogenates were conducted and 0.1 mL aliquots were spread on SDA plates and the plates incubated at 35° C. overnight. The CFU/kidneys were determined from colony counts. Data were analyzed using one-way ANOVA with the Tukey-Kramer Multiple Comparisons Test (GraphPad InStat version 3.06, GraphPad Software, San Diego, Calif.).

First Run

The data tabulated in Table 2 shows the mean density of C. albicans in kidneys of mice treated with test compounds 2 hr after infection and kidneys evaluated at 24 hr after infection. Treatments were administered at 2 hr after infection with 2.1×10⁵ CFU/mL of the organism. Data are the means of four mice.

TABLE 2

| | Mean log₁₀ | |
|---|---|---|
| | CFU/kidneys | Difference from |
| Treatment | (±SD) | 24-hr control |
| Infected control - 2 h | 3.01 ± 0.08 | |
| Infected control - 24 h | 4.77 ± 0.15 | |
| anidulafungin - 1 mg/kg | 3.09 ± 0.61 | −1.68*** |
| caspofungin - 1 mg/kg | 1.63 ± 0.29 | −3.14*** |
| Compound 1 - 1 mg/kg | 4.65 ± 0.26 | −0.13 ns |
| Compound 1 - 5 mg/kg | 1.90 ± 0.21 | −2.87*** |
| Compound 2 - 1 mg/kg | 5.10 ± 0.28 | 0.33 ns |
| Compound 2 - 5 mg/kg | 5.14 ± 0.25 | 0.37 ns |
| Compound 3&11 (1:1) - 1 mg/kg | 5.26 ± 0.25 | 0.49 ns |
| Compound 3&11 (1:1) - 5 mg/kg | 5.01 ± 0.38 | 0.24 ns |
| Compound 4 - 1 mg/kg | 5.25 ± 0.09 | 0.48 ns |
| Compound 4 - 5 mg/kg | 5.37 ± 0.11 | 0.60 ns |
| Compound 5 - 1 mg/kg | 4.77 ± 0.06 | 0.00 ns |
| Compound 5 - 5 mg/kg | 3.01 ± 0.41 | −1.76*** |
| Compound 7 - 1 mg/kg | 5.27 ± 0.17 | 0.50 ns |
| Compound 7 - 5 mg/kg | 5.08 ± 0.19 | 0.31 ns |

ns, difference not significant;
***significant at P < 0.001.

Results from the First Run

The density of C. albicans in the infected control mice at 2 hr after infection was 3.01 logs, and increased to 4.77 logs by 24 hr after infection (Table 2). Two of the test compound treatments also significantly reduced yeast density. Compound 1 and compound 5, each at the 5 mg/kg dose, reduced the density by 2.87 and 1.76 logs, respectively.

Second Run

The data tabulated in Table 3 shows the mean density of C. albicans in kidneys of mice treated with test compounds 2 hr after infection and kidneys evaluated at 24 hr after infection. Treatments were administered at 2 hr after infection with 6.4×10⁵ CFU/mL of the organism. Data are the means of four mice.

TABLE 3

| | Mean log₁₀ | |
|---|---|---|
| | CFU/kidneys | Difference from |
| Treatment | (±SD) | 24-hr control |
| Infected control - 2 h | 3.27 ± 0.12 | |
| Infected control - 24 h | 5.20 ± 0.22 | |
| anidulafungin - 1 mg/kg | 3.37 ± 0.72 | −1.83*** |
| caspofungin - 1 mg/kg | 1.61 ± 0.23 | −3.59*** |
| Compound 18 - 5 mg/kg | 1.76 ± 0.44 | −3.44*** |
| Compound 19 - 5 mg/kg | 1.71 ± 0.35 | −3.49*** |
| Compound 20 - 5 mg/kg | 1.93 ± 0.44 | −3.27*** |
| Compound 12 - 5 mg/kg | 4.42 ± 0.14 | −0.78 ns |
| Compound 13 - 5 mg/kg | 0.65 ± 0.75 | −4.55*** |
| Compound 14 - 5 mg/kg | 4.07 ± 0.17 | −1.13 ns |
| Compound 15 - 5 mg/kg | 4.75 ± 0.20 | −0.45 ns |

ns, difference not significant;
***significant at P < 0.001.

Results from the Second Run

The density of C. albicans in the infected control mice at 2 hr after infection was 3.27 logs, and increased to 5.20 logs by 24 hr after infection (Table 3). All of the test compounds, with the exception of compound 12, compound 14, and compound 15, significantly reduced yeast density in the kidneys of infected mice compared to the infected controls.

Third Run

The data tabulated in Table 4 shows the mean density of C. albicans in kidneys of mice treated with test compounds 2 hr after infection and kidneys evaluated at 24 hr after infection. Treatments were administered at 2 hr after infection with 3.8×10⁵ CFU/mL of the organism. Data are the means of four mice.

TABLE 4

| | Mean log₁₀ | Difference from |
|---|---|---|
| | CFU/kidneys | 24-hr |
| Treatment | (±SEM) | control |
| Infected control - 2 h | 2.92 ± 0.07 | |
| Infected control - 24 h | 4.87 ± 0.10 | |
| anidulafungin - 0.5 mg/kg | 4.41 ± 0.12 | −0.47 ns |
| anidulafungin - 1.5 mg/kg | 2.77 ± 0.20 | −2.11*** |
| anidulafungin - 4.5 mg/kg | 1.10 ± 0.38 | −3.78*** |
| Compound 19 - 0.5 mg/kg | 4.84 ± 0.26 | −0.03 ns |
| Compound 19 - 1.5 mg/kg | 2.82 ± 0.10 | −2.06*** |
| Compound 19 - 4.5 mg/kg | 1.05 ± 0.36 | −3.82*** |
| Compound 21 - 0.5 mg/kg | 3.35 ± 0.08 | −1.53** |
| Compound 21 - 1.5 mg/kg | 2.26 ± 0.12 | −2.62*** |
| Compound 21 - 4.5 mg/kg | 0.65 ± 0.38 | −4.22*** |

ns, difference not significant;
**significant at P < 0.01;
***significant at P < 0.001.

Results from the Third Run

The density of C. albicans in the infected control mice at 2 hr after infection was 2.92 logs, and increased to 4.87 logs by 24 hr after infection (Table 4). All dose levels of compound 21 significantly reduced the density of C. albicans in the kidneys of treated mice compared to the infected controls.

Fourth Run

The data tabulated in Table 5 shows the mean density of C. albicans in kidneys of mice treated with test compounds 2 hr after infection and kidneys evaluated at 24 hr after infection. Treatments were administered at 2 hr after infection with 3.8×10⁵ CFU/rnL of the organism. Data are the means of four mice.

TABLE 5

| | Mean log₁₀ | Difference from |
|---|---|---|
| Treatment | CFU/kidneys | 24-h |
| (mg/kg) | (±SEM) | control |
| Infected control - 2 h | 2.89 ± 0.07 | |
| Infected control - 24 h | 5.20 ± 0.07 | |
| caspofungin - 0.5 mg/kg | 0.33 ± 0.33 | −4.88*** |
| caspofungin - 1.5 mg/kg | 0.73 ± 0.42 | −4.48*** |
| caspofungin - 4.5 mg/kg | 0.00 ± 0.00 | −5.20*** |
| Compound 16 - 0.5 mg/kg | 5.05 ± 0.12 | −0.16 ns |
| Compound 16 - 1.5 mg/kg | 5.16 ± 0.03 | −0.04 ns |
| Compound 16 - 4.5 mg/kg | 4.78 ± 0.18 | −0.42 ns |
| Compound 17 - 0.5 mg/kg | 4.33 ± 0.14 | −0.87 ns |
| Compound 17 - 1.5 mg/kg | 3.11 ± 0.14 | −2.09*** |
| Compound 17 - 4.5 mg/kg | 0.95 ± 0.55 | −4.25*** |

ns, difference not significant;
***significant at P < 0.001.

Results from the Fourth Run

The density of C. albicans in the infected control mice at 2 hr after infection was 2.89 logs, and increased to 5.20 logs by 24 hr after infection (Table 5). Compound 17 significantly reduced the density at 4.5 and 1.5 mg/kg, but not at 0.5 mg/kg. Compound 16 was ineffective in this assay at all dose levels.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. An echinocandin class compound comprising a PEG, alkyl-PEG, aryl-PEG, alkaryl-PEG, PEG-alkyl, PEG-aryl, or PEG-alkaryl group.

2. The echinocandin class compound of claim 1, wherein said echinocandin class compound is further described by formula (I): 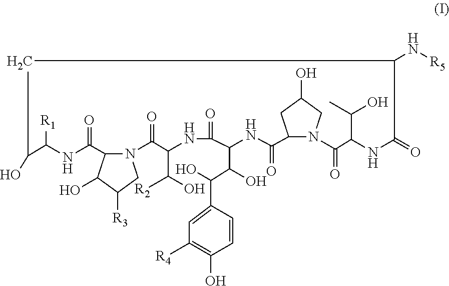
wherein,
R¹ is NHCH₂CH₂NHR^{A1}, NHCH₂CH₂NR^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1};
R² is H, CH₃, CH₂CH₂NHR^{B1}, CH₂CH₂NR^{B1}R^{B2}, CH₂CH₂NHC(O)R^{B1}, CH₂C(O)NHR^{B1}, CH₂CH₂CH(OR^{B1})NHR^{B2}, CH₂CH₂CH(OR^{B1})NR^{B2}R^{B3}, or CH₂CH₂CH(OR^{B1})NHC(O)R^{B2};
R³ is H or CH₃;
R⁴ is H, OSO₃H, CH₂NHR^{C1}, CH₂NR^{C1}R^{C2}, CH₂NHC(O)R^{C1};
R⁵ is a lipophilic group selected from: PEG; C(O)-PEG; PEG-alkyl; C(O)-PEG-alkyl; PEG-aryl; C(O)-PEG-aryl; PEG-alkaryl; C(O)-PEG-alkaryl; alkyl-PEG; C(O)-alkyl-PEG; aryl-PEG; C(O)-aryl-PEG; alkaryl-PEG;
C(O)-alkaryl-PEG; 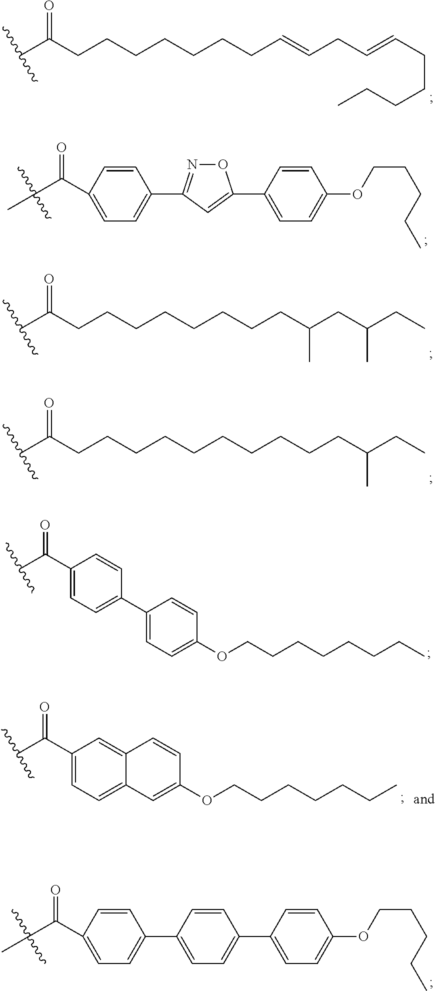
each of R^{A1}, R^{A2}, R^{B1}, R^{B2}, R^{B3}, R^{C1}, and R^{C2} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl,
and pharmaceutically acceptable salts thereof, provided that said echinocandin class compound comprises at least one PEG, alkyl-PEG, aryl-PEG, alkaryl-PEG, PEG-alkyl, PEG-aryl, or PEG-alkaryl group.

3. The echinocandin class compound of claim 1, wherein said echinocandin class compound is further described by formula (II): 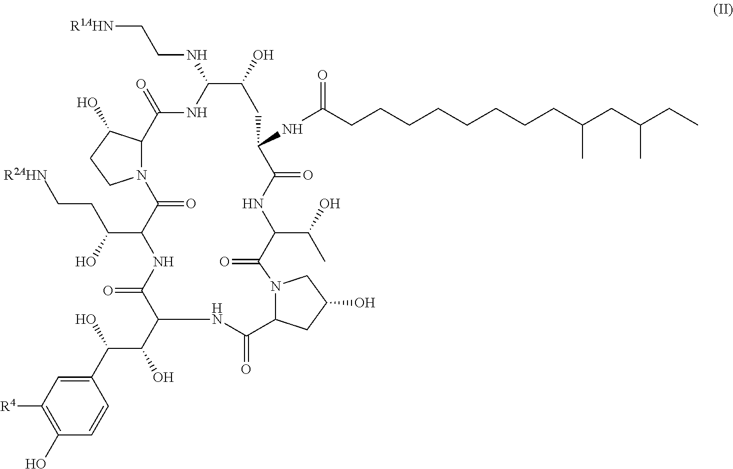
wherein,
R^{1A} is H, C₁₋₁₀ alkyl, C₂₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, or PEG-alkaryl;
R^{2A} is H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, or PEG-alkaryl;
R⁴ is H, OSO₃H, CH₂NHR^{C1}, CH₂NR^{C1}R^{C2}, CH₂NHC(O)R^{C1}; and
each of R^{C1} and R^{C2} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl,
and pharmaceutically acceptable salts thereof, provided that said echinocandin class compound comprises at least one PEG, alkyl-PEG, aryl-PEG, alkaryl-PEG, PEG-alkyl, PEG-aryl, or PEG-alkaryl.

4. The echinocandin class compound of claim 3, wherein one of R^{1A}, R^{2A}, R^{C1} and R^{C2} is selected from:
(i) —(CH₂)ₚ—O—(CH₂CH₂O)ₘ—Me, and
(ii) —(CH₂CH₂O)ₘ—Me, and
(iii) —C(O)(CH₂)ₙ—(OCH₂CH₂)ₘ—OMe,
wherein n is an integer from 0 to 11, p is an integer from 3 to 12, and m is an integer from 1 to 10.

5. The echinocandin class compound of claim 1, wherein said echinocandin class compound is further described by formula (III): 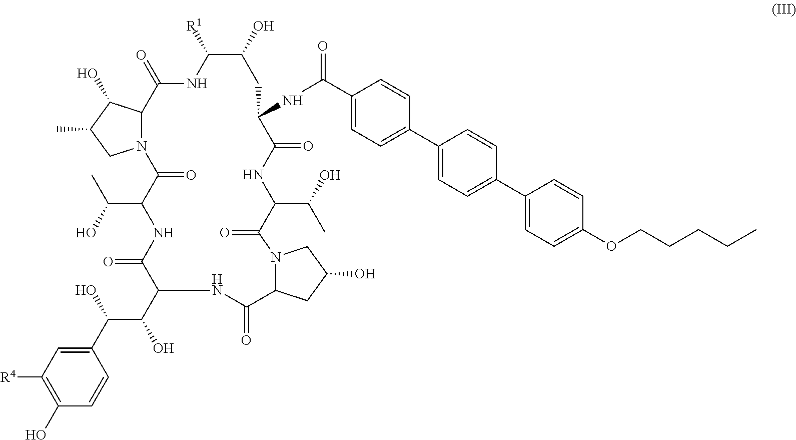
wherein,
R¹ is NHCH₂CH₂NHR^{A1}, NHCH₂CH₂NR^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1};
R⁴ is H, OSO₃H, CH₂NHR^{C1}, CH₂NR^{C1}R^{C2}, CH₂NHC(O)R^{C1}; and
each of R^{A1}, R^{A2}, R^{C1}, and R^{C2} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl,
and pharmaceutically acceptable salts thereof, provided that said echinocandin class compound comprises at least one PEG, alkyl-PEG, aryl-PEG, alkaryl-PEG, PEG-alkyl, PEG-aryl, or PEG-alkaryl group.

6. The echinocandin class compound of claim 5, wherein R¹ is selected from:
(i) —O—(CH₂CH₂O)ₘ—(CH₂)ₙ—Me,
(ii) —NH—(CH₂CH₂O)ₘ—(CH₂)ₙ—Me,
(iii) —O—(CH₂)_{q}—O—(CH₂CH₂O)ₘ—Me,
(iv) —NH—(CH₂)_{q}—O—(CH₂CH₂O)ₘ—Me,
(v) —O—(CH₂)ₚ—NH—(CO)—(CH₂)ₙ—O—(CH₂CH₂O)ₘ—Me,
(vi) —NH—(CH₂)ₚ—NH—(CO)—(CH₂)ₙ—O—(CH₂CH₂O)ₘ—Me,
(vii) —NHCH[(CH₂O(CH₂CH₂O)ₛ—Me)(CH₂O(CH₂CH₂O)ₜ—Me)], and
(viii) —O—CH[(CH₂O(CH₂CH₂O)ₛ—Me)(CH₂O(CH₂CH₂O)ₜ—Me)],
wherein n is an integer from 0 to 11, q is an integer from 3 to 12, p is an integer from 2 to 8, s is an integer from 0 to 5, t is an integer from 0 to 5, and m is an integer from 1 to 10.

7. The echinocandin class compound of claim 1, wherein said echinocandin class compound is further described by formula (IV): 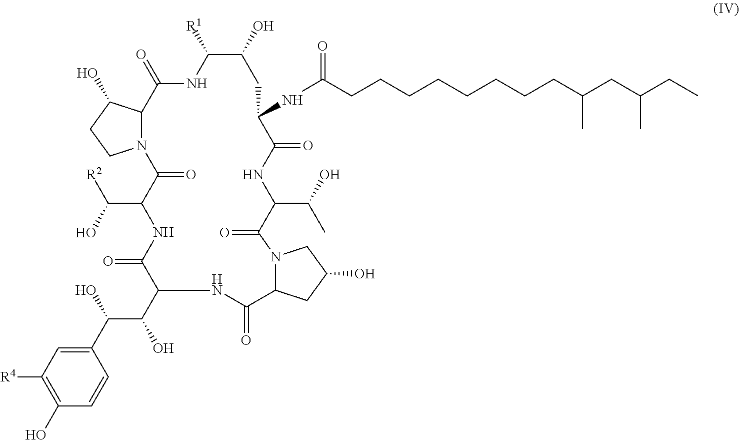
wherein,
R¹ is NHCH₂CH₂NHR^{A1}, NHCH₂CH₂NR^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1};
R² is H, CH₃, CH₂CH₂NHR^{B1}, CH₂CH₂NR^{B1}R^{B2}, CH₂CH₂NHC(O)R^{B1}, CH₂C(O)NHR^{B1}, CH₂CH₂CH(OR^{B1})NHR^{B2}, CH₂CH₂CH(OR^{B1})NR^{B2}R^{B3}, or CH₂CH₂CH(OR^{B1})NHC(O)R^{B2};
R⁴ is H, OSO₃H, CH₂NHR^{C1}, CH₂NR^{C1}R^{C2}, CH₂NHC(O)R^{C1l ; and}
each of _{R}^{A1}, R^{A2}, R^{B1}, R^{B2}, R^{B3}, R^{C1}, and R^{C2} is, independently, selected from H, C₁₋₁₀ alkyl, C₂₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl,
and pharmaceutically acceptable salts thereof, provided that said echinocandin class compound comprises at least one PEG, alkyl-PEG, aryl-PEG, alkaryl-PEG, PEG-alkyl, PEG-aryl, or PEG-alkaryl group.

8. The echinocandin class compound of claim 7, wherein R¹ is selected from:
(i) —O—(CH₂CH₂O)ₘ—(CH₂)ₙ—Me,
(ii) —NH—(CH₂CH₂O)ₘ—(CH₂)ₙ—Me,
(iii) —O—(CH₂)_{q}—O—(CH₂CH₂O)ₘ—Me,
(iv) —NH—(CH₂)_{q}—O—(CH₂CH₂O)ₘ—Me,
(v) —O—(CH₂)ₚ—NH—(CO)—(CH₂)ₙ—O—(CH₂CH₂O)ₘ—Me,
(vi) —NH—(CH₂)ₚ—NH—(CO)—(CH₂)ₙ—O—(CH₂CH₂O)ₘ—Me,
(vii) —NHCH[(CH₂O(CH₂CH₂O)ₛ—Me)(CH₂O(CH₂CH₂O)ₜ—Me)], and
(viii) —O—CH[(CH₂O(CH₂CH₂O)ₛ—Me)(CH₂O(CH₂CH₂O)ₜ—Me)],
wherein n is an integer from 0 to 11, q is an integer from 3 to 12, p is an integer from 2 to 8, s is an integer from 0 to 5, t is an integer from 0 to 5, and m is an integer from 1 to 10.

9. The echinocandin class compound of claim 1, wherein said echinocandin class compound is further described by formula (V): 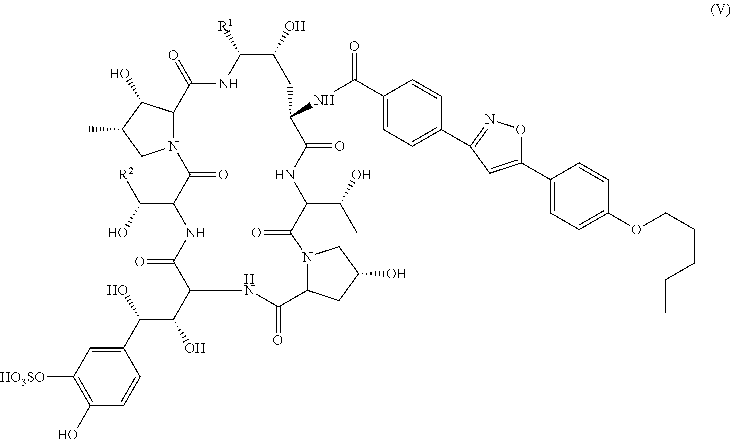
wherein,
R¹ is NHCH₂CH₂NHR^{A1}, NHCH₂CH₂NR^{A1}R^{A2}, NHCH₂CH₂NHC(O)R^{A1}, CH₂NHR^{A1}, CH₂NR^{A1}R^{A2}, CH₂NHC(O)R^{A1}, or OR^{A1};
R² is H, CH₃, CH₂CH₂NHR^{B1}, CH₂CH₂NR^{B1}R^{B2}, CH₂CH₂NHC(O)R^{B1}, CH₂C(O)NHR^{B1}, CH₂CH₂CH(OR^{B1})NHR^{B2}, CH₂CH₂CH(OR^{B1})NR^{B2}R^{B3}, or CH₂CH₂CH(OR^{B1})NHC(O)R^{B2}; and
each of R^{A1}, R^{A2}, R^{B1}, R^{B1}, and R^{B3} is, independently, selected from H, C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, C₁₋₁₀ heteroalkyl, PEG, alkyl-PEG, aryl-PEG, alkaryl-PEG, PEG-alkyl, PEG-aryl, and PEG-alkaryl,
and pharmaceutically acceptable salts thereof, provided that said echinocandin class compound comprises at least one PEG, alkyl-PEG, aryl-PEG, alkaryl-PEG, PEG-alkyl, PEG-aryl, or PEG-alkaryl group.

10. The echinocandin class compound of claim 9, wherein R¹ is selected from:
(i) —O—(CH₂CH₂O)ₘ—(CH₂)ₙ—Me,
(ii) —NH—(CH₂CH₂O)ₘ—(CH₂)ₙ—Me,
(iii) —O—(CH₂)_{q}—O—(CH₂CH₂O)ₘ—Me,
(iv) —NH—(CH₂)_{q}—O—(CH₂CH₂O)ₘ—Me,
(v) —O—(CH₂)ₚ—NH—(CO)—(CH₂)ₙ—O—(CH₂CH₂O)ₘ—Me,
(vi) —NH—(CH₂)ₚ—NH—(CO)—(CH₂)ₙ—O—(CH₂CH₂O)ₘ—Me,
(vii) —NHCH[(CH₂O(CH₂CH₂O)ₛ—Me)(CH₂O(CH₂CH₂O)ₜ—Me)], and
(viii) —O—CH[(CH₂O(CH₂CH₂O)ₛ—Me)(CH₂O(CH₂CH₂O)ₜ—Me)],
wherein n is an integer from 0 to 11, q is an integer from 3 to 12, p is an integer from 2 to 8, s is an integer from 0 to 5, t is an integer from 0 to 5, and m is an integer from 1 to 10.

11. The echinocandin class compound of claim 2, 3, 5, or 7 wherein R⁴ is selected from:
(i) —CH₂NH—(CH₂CH₂O)ₘ—(CH₂)ₙ—Me,
(ii) —CH₂NH—(CH₂)_{q}—O—(CH₂CH₂O)ₘ—Me,
(iii) —CH₂NH—(CH₂)ₚ—NH—(CO)—(CH₂)ₙ—O—(CH₂CH₂O)ₘ—Me, and
(iv) —CH₂NHCH[(CH₂O(CH₂CH₂O)ₛ—Me)(CH₂O(CH₂CH₂O)ₜ—Me)],
wherein n is an integer from 0 to 11, q is an integer from 3 to 12, p is an integer from 2 to 8, s is an integer from 0 to 5, t is an integer from 0 to 5, and m is an integer from 1 to 10.

12. The echinocandin class compound of claim 2, wherein R⁵ is selected from:
(i) —(CH₂CH₂O)ₘ—(CH₂)ₙ—Me,
(ii) —C(O)—(CH₂CH₂O)ₘ—(CH₂)ₙ—Me,
(iii) —C(O)CH₂—O—(CH₂CH₂O)ₘ—(CH₂)ₙ—Me, and
(iv) —C(O)—O—(CH₂CH₂O)ₘ—(CH₂)ₙ—Me,
wherein n is an integer from 0 to 11, and m is an integer from 1 to 10.

13. The echinocandin class compound of claim 1, wherein said echinocandin class compound has increased oral bioavailability; has increased transdermal bioavailability; or has an increased therapeutic index.

14. (canceled)

15. (Canceled)

16. A pharmaceutical composition comprising an echinocandin class compound of claim 1, or a salt thereof, and a pharmaceutically acceptable excipient.

17. The pharmaceutical composition of claim 16, wherein said pharmaceutical composition comprising a mixture of echinocandin class compounds which are substantially monodisperse.

18. The pharmaceutical composition of claim 16, wherein said pharmaceutical composition is formulated for oral administration in unit dosage form.

19. A method of treating a fungal infection in a subject, said method comprising administering to said subject an echinocandin class compound of claim 1, or a salt thereof, in an amount sufficient to treat said infection.

20. The method of claim 19, wherein said infection is selected from tinea capitis, tinea corporis, tinea pedis, onychomycosis, perionychomycosis, pityriasis versicolor, oral thrush, vaginal candidosis, respiratory tract candidosis, biliary candidosis, eosophageal candidosis, urinary tract candidosis, systemic candidosis, mucocutaneous candidosis, aspergillosis, mucormycosis, paracoccidioidomycosis, North American blastomycosis, histoplasmosis, coccidioidomycosis, or sporotrichosis.

21. The method of claim 19, wherein wherein said fungal infection is an infection of Candida albicans, C. parapsilosis, C. glabrata, C. guillierrnondii, C. krusei, C. tropicalis, Aspergillus fumigatus, A. flavus, or A. terreus.

22. The method of claim 19, wherein said echinocandin class compound is administered orally.

23. A method of preventing, stabilizing, or inhibiting the growth of fungi, or killing fungi, said method comprising contacting said fungi or a site susceptible to fungal growth with an echinocandin class compound of claim 1, or a salt thereof.
